(19) United States
(12) Patent Application Publication
(10) Pub. No.: US 2025/0302313 A1
(43) Pub. Date: Oct. 2, 2025

(54) PHOTOACOUSTIC IMAGING FOR NONINVASIVE PERIODONTAL PROBING DEPTH MEASUREMENTS

(71) Applicant: The Regents of the University of California, Oakland, CA (US)

(72) Inventors: Jesse JOKERST, San Diego, CA (US); Colman MOORE, San Diego, CA (US); Ali HARIRI, San Diego, CA (US)

(21) Appl. No.: 18/229,137

(22) Filed: Aug. 1, 2023

Related U.S. Application Data
(63) Continuation of application No. 16/635,115, filed on Jan. 29, 2020, now abandoned.
(60) Provisional application No. 62/561,965, filed on Sep. 22, 2017.

(51) Int. Cl. A61B 5/00

(52) U.S. Cl. CPC A61B 5/0088; A61B 5/0062; A61B 5/0095; A61B 5/682; A61B 5/4552

(57) ABSTRACT

In alternative embodiments, provided are methods and products of manufacture for photoacoustic imaging of the periodontium of individuals in need thereof for noninvasive periodontal probing depth measurements and periodontal pocket imaging and gingival thickness. In alternative embodiments, products of manufacture as provided herein can observe or measure soft tissue contrast, e.g., gums, the periodontium, or the oral mucosa can be imaged and/or measured.

### RELATED APPLICATIONS

This application is a continuation of U.S. Utility application Ser. No. 16/635,115, filed Jan. 29, 2020, now pending, which is a national phase application claiming benefit of priority under 35 U.S.C. § 371 to Patent Convention Treaty (PCT) International Application PCT/US2018/052270, filed on Sep. 21, 2018, now expired, which claims the benefit of priority to U.S. Provisional Application Ser. No. (USSN) 62/561,965, Sep. 22, 2017, now expired. The aforementioned applications are expressly incorporated herein by reference in their entirety and for all purposes.

### STATEMENT AS TO FEDERALLY SPONSORED RESEARCH

This invention was made with government support under grant nos. DP2 HL137187 and S10 OD021821. The government has certain rights in the invention.

### TECHNICAL FIELD

This invention generally relates to dentistry and dental materials. In alternative embodiments, provided are methods and products of manufacture for photoacoustic imaging of the periodontium of individuals in need thereof for noninvasive periodontal probing depth measurements and periodontal pocket imaging and gingival thickness. In alternative embodiments, products of manufacture as provided herein can observe or measure soft tissue contrast, e.g., gums, or the periodontium or the oral mucosa can be imaged and/or measured, including hypoxia and inflammation/infection.

### BACKGROUND

Periodontitis affects nearly 50% of Americans and exerts both local and systemic effects on the body. These range from mild discomfort to debilitating pain, tooth loss, and excessive activation of the immune system. Studies have identified the chronic inflammation from periodontitis as a risk factor for conditions such as cardiovascular disease, cancer, and dementia. Thus, it is critical to diagnose periodontal disease early while the symptoms are mild and reversible.

Current metrics for monitoring periodontal health include attachment level, probing depth, bone loss, mobility, recession, and degree of inflammation. For example, a periodontal examination evaluates the periodontium for signs of inflammation or damage and is the basis for subsequent intervention. One key feature of this assessment is the measurement of probing depths, which is a numeric metric that reflects the extent of apical epithelial attachment relative to the gingival margin and is critical for disease staging. Probing depth measurements can identify periodontal disease and monitor response to intervention. The probe depths offer insight into clinical attachment loss, furcation involvement, and bone loss when used in conjunction with radiography and the oral examination.

Probing depths are commonly measured with a periodontal probe, which has remained popular despite the advent of many next-generation tools. Unfortunately, the periodontal probe is a relatively unsophisticated tool. Probing depth measurements using the periodontal probe are error prone and suffer from poor reproducibility, largely due to variation in probing force. Indeed, a recent meta-analysis showed that a range of probing forces were used, which varied across multiple orders of magnitude. Other error sources include variation in the insertion point, probe angulation, the patient's overall gingival health (weakly inflamed tissue), and the presence of calculus. Thus, the examination is subject to large errors with interoperator variation as high as 40% and r values <0.80. Furthermore, the probe can only measure depth at the point of insertion, with no information on the full width or contour of the pocket.

These error sources can result in poor patient treatment and, hence, poor patient outcomes. The variation also compromises epidemiologic studies and makes it difficult to compare outcomes among dentists or among populations. Furthermore, the probe often penetrates the inflamed epithelium, resulting in patient discomfort, bleeding on probing, and producing probe depths that are up to 1 mm deeper than the actual anatomic value.

While some studies have shown that a constant force probe or digital probe might overcome most limitations, such tools underestimate probing with little improvement in reproducibility. Clearly, new tools, including improved imaging tools, are urgently needed to improve this vital procedure.

Ultrasound is an affordable, high-resolution, sensitive, nonionizing, and real-time tool for imaging, but it is rarely used in dentistry. Previous studies have used ultrasound with frequencies ≤20 MHz to image facial crestal bone or the cementoenamel junction, but these approaches lacked the spatial resolution and contrast needed to measure probing depth.

More recently, photoacoustic imaging has been used in addition to ultrasound to combine the temporal and spatial resolution of acoustics with the spectral behavior and increased contrast of optics. In addition, the use of high frequency offers <100-μm resolution to image the probing depths.

### SUMMARY

In alternative embodiments, provided are products of manufacture or dental devices for imaging a periodontal tissue or a periodontium, wherein optionally the periodontal tissue comprises an oral mucosa or gingiva, the method comprising:
a mouthpiece or mold capable of fitting over one or several teeth, or all teeth, in an arch (optionally a maxillary or mandibular arch, or both), and optionally also fitting over or covering (e.g., at least part of) the periodontal tissue or periodontium,
wherein optionally the mouthpiece or mold is manufactured to: image and collect signal(s) from a contrasting or imaging agent, and/or, to hold, carry and deliver to the contrasting agent or imaging agent to the periodontal tissue or periodontium, when the mouthpiece or mold is placed or fitted over the one or several or all teeth in the arch, optionally also covering periodontal tissue or periodontium or gingiva, and
one or more photoacoustic imaging sensors and corresponding transducers, wherein the one or more photoacoustic imaging sensors comprise ultrasound and/or optical imaging sensors capable of generating imaging data, and the ultrasound and optical imaging sensors are operatively linked to the corresponding ultrasound and optical transducers, and the ultrasound and/or optical transducer is capable of transmitting the imaging data to a remote receiver,
wherein the one or more photoacoustic imaging sensors can image the periodontium or periodontal tissue, or a periodontal pocket, and generate imaging data,
and optionally the imaging data from the one or more photoacoustic imaging sensors is capable of being converted to: an image of the periodontium or periodontal tissue, or an image of the periodontal pocket, or a periodontal probing depth image or measurement.

In alternative embodiments, of the products of manufacture or dental devices as provided herein:
wherein the mouthpiece or mold comprises an outer body shaped to fit over (or cover) the one or several teeth, or all teeth, optionally also including fitting over or covering (e.g., at least part of) the periodontium or gingiva, in an arch;
the one or more photoacoustic imaging sensors and corresponding transducers comprises: one or more ultrasonic transducers disposed within or on the outer body;
the one or more ultrasonic transducers are disposed within or on the outer body to substantially align with the one or several teeth, or all teeth, in an arch when the outer body is inserted into a patient's mouth;
the one or more photoacoustic imaging sensors and corresponding transducers further comprises one or more light generators (e.g., at any wavelength, including visible or UV, for example), optionally light-emitting diodes (LEDs), disposed within or on the outer body;
the one or more light generators are received within openings in a gum-facing surface of the outer body;
the products of manufacture or dental devices further comprise a wired or wireless transmittal device to transmit the imaging data to a computer and/or a database, or to an electronic medical record or to a computer hard disk or computer memory, or to a cloud-based storage source, and optionally the ultrasound and/or optical transducers operatively linked to their corresponding ultrasound and optical imaging sensors are operatively linked to the wired or wireless transmittal device;
the mouthpiece or mold comprises an outer body shaped to fit over the one or several teeth, or all teeth, in an arch; and further comprising a non-transitory memory stored within an outer body of the mouth and configured for storing the imaging data;
the products of manufacture or dental devices further comprise an acoustic coupling medium or a gel, or equivalent, to transmit acoustic pressure waves from an oral mucosa, a gum, a tooth or a periodontal pocket, to the acoustic transducers in the product of manufacture or dental device;
the products of manufacture or dental devices further comprise a photoacoustic imaging agent, wherein optionally photoacoustic imaging agent comprises a food based imaging agent, or optionally comprises a food-grade squid ink or an equivalent optionally also comprising a cornstarch or an equivalent, and optionally photoacoustic imaging agent comprises an optical absorber, optionally a strong optical absorber, or optionally an optical absorber comprising a melanin; and/or,
the photoacoustic imaging agent comprises a plurality of melanin nanoparticles.

In alternative embodiments products of manufacture or dental devices as provided herein do not comprise a photoacoustic imaging agent, but rather a photoacoustic signal is generated via hemoglobin and deoxyhemoglobin in the gingiva or periodontal pocket. This vascular imaging can indicate the degree of inflammation or infection.

In alternative embodiments, provided are methods for:
measuring or detecting an inflammation or a bacterial infection, or a hypoxia or blood flow (e.g., the amount of blood flow), in an oral mucosa, a periodontal pocket or in a periodontium (e.g., gingiva),
image a periodontium or periodontal tissue, or a periodontal pocket,
measuring the depth of a periodontal pocket,
measuring or detecting calcification, calculus (or tartar, or hardened dental plaque) or dental stains,
measuring or detecting gingival thickness, and/or
measuring or detecting peri-implantitis, comprising:
(a) providing a product of manufacture or a dental device of any of the preceding claims;
(b) providing a photoacoustic imaging agent, and/or an acoustic coupling medium or a gel, or equivalent,
wherein optionally photoacoustic imaging agent comprises a food based imaging agent, or optionally comprises a food-grade squid ink or an equivalent optionally also comprising a cornstarch or an equivalent,

wherein optionally photoacoustic imaging agent comprises an optical absorber, optionally a strong optical absorber, or optionally an optical absorber comprising a melanin,
(c) placing the photoacoustic imaging agent and/or acoustic coupling medium or a gel, or equivalent on the periodontium, gingiva, or the oral mucosa of an individual, optionally a dental patient, or placing the photoacoustic imaging agent and/or acoustic coupling medium or a gel, or equivalent in the product of manufacture or the dental device such that the photoacoustic imaging agent comes into contact with the periodontium or oral mucosa,
(d) taking a photoacoustic and/or acoustic coupling medium or a gel, or equivalent imaging of the periodontium or oral mucosa with the one or more photoacoustic imaging sensors to generate ultrasound and/or optical imaging data, and
optionally, (e) saving the ultrasound and/or optical imaging data in the product of manufacture or the dental device, wherein the product of manufacture or a dental device comprises a data memory storage, or transmitting the ultrasound and optical imaging data to a computer, a memory and/or a database.

In alternative embodiments of methods as provided herein:
the methods further comprise (or comprise use of) a wired or wireless transmittal device to transmit the imaging data to a computer and/or a database, or to an electronic medical record or to a computer hard disk or computer memory, or to a cloud-based storage source; and/or
the ultrasound and/or optical imaging data from the one or more photoacoustic imaging sensors is capable of being converted to: an image of the periodontium or periodontal tissue, or an image of a periodontal pocket, or a periodontal probing depth measurement, or the ultrasound and optical imaging data from the one or more photoacoustic imaging sensors is converted to: an image of the periodontium or periodontal tissue, or an image of a periodontal pocket, or a periodontal probing depth measurement.

In alternative embodiments provided are Uses of a product of manufacture or dental device of any of the preceding claims for:
measuring or detecting an inflammation or a bacterial infection, or a hypoxia or blood flow (e.g., the amount of blood flow), in an oral mucosa, a periodontal pocket or in a periodontium (e.g., gingiva),
image a periodontium or periodontal tissue, or a periodontal pocket,
measuring the depth of a periodontal pocket,
measuring or detecting calcification, calculus (or tartar, or hardened dental plaque) or dental stains,
measuring or detecting gingival thickness, and/or
measuring or detecting peri-implantitis.

In alternative embodiments provided are products of manufacture or dental devices (as provided herein) for use in:
measuring or detecting an inflammation or a bacterial infection, or a hypoxia or blood flow (e.g., the amount of blood flow), in an oral mucosa, a periodontal pocket or in a periodontium (e.g., gingiva),
image a periodontium or periodontal tissue, or a periodontal pocket,
measuring the depth of a periodontal pocket,
measuring or detecting calcification, calculus (or tartar, or hardened dental plaque) or dental stains,
measuring or detecting gingival thickness, and/or
measuring or detecting peri-implantitis.

In alternative embodiments provided are kits comprising a product of manufacture or dental device of any of the preceding claims, and optionally further comprising a photoacoustic imaging agent and/or acoustic coupling medium or a gel, or equivalent, wherein optionally photoacoustic imaging agent comprises a food based imaging agent, or optionally comprises a food-grade squid ink or an equivalent optionally also comprising a cornstarch or an equivalent, wherein optionally photoacoustic imaging agent comprises an optical absorber, optionally a strong optical absorber, or optionally an optical absorber comprising a melanin, and optionally further comprising instructions for practicing a method of any of the preceding claims.

In alternative embodiments, kits as provided herein further comprise:
an applicator for applying the photoacoustic imaging agent to a patient's mouth, wherein optionally the applicator is a syringe, and optionally the syringe is preloaded with the photoacoustic imaging agent; and/or
a processing device for receiving the imaging data and generating at least one image of the periodontal tissue or periodontium, and optionally further comprising an imaging device for displaying the image of the periodontal tissue or periodontium.

In alternative embodiments of kits as provided herein:
said processing device comprises:
a processor;
a wired or wireless interface for operably communicating, directly or indirectly, with the photoacoustic imaging sensors;
computer-executable instructions stored on a non-transitory memory for causing the processor to receive the image data and generate the at least one image of the periodontal tissue or periodontium;
said processing device is further configured to:
generate an additional ultrasound image of the periodontal tissue or periodontium, or portion thereof; and
overlay one of the generate the at least one image of the periodontal tissue or periodontium or the generated additional ultrasound image, or vice versa, to produce a composite image.

In alternative embodiments, kits as provided herein further comprise: an additional processing device for receiving the imaging data directly from the photoacoustic imaging sensors, storing at least a portion of the receiving imaging data, and transmitting the stored imaging data, wired and/or wirelessly, to the processing device.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

Figures are described in detail herein.

FIG. 1A-F illustrate: Characterization of cuttlefish ink and photoacoustic signal of cuttlefish ink/cornstarch contrast agent used in an example method.

FIG. 1A illustrates Transmission electron microscopy image of 0.5% ink A illustrating spherical melanin nanoparticles found in cuttlefish ink.

FIG. 1B illustrates The hydrodynamic radius of melanin nanoparticles in phosphate buffered saline (PBS) by dynamic light scattering was 266.3 nm, with a polydispersity index of 0 116; the insert figure is the measurement of 500 nanoparticles on transmission electron microscopy images, indicating a mean size of

FIG. 1C illustrates Photoacoustic spectra of inks A (top line) and B (middle line) were studied at 1% in 0.1M PBS (bottom line).

FIG. 1D illustrates Overlaid ultrasound mode (grayscale) and photoacoustic (red) images of a phantom with cuttlefish ink/cornstarch contrast agent with 2% cornstarch and 10%, 5%, 1%, and 0% cuttlefish ink. 125.1 nm.

FIG. 1E illustrates Imaging data as a function of pH show that the photoacoustic signal of the example contrast agent with 5% cuttlefish ink and 2% cornstarch was stable from pH 6.2 to 7.8.

FIG. IF illustrates Photoacoustic intensity as a function of depth was performed for a cuttlefish ink/cornstarch contrast agent with 5% cuttlefish ink and 2% cornstarch, except for the uppermost sample (PBS control). The signal 11 mm from the transducer head was >4-fold higher than that at 11.8 mm. For panels E and F, the error bars represent the standard deviation of 8 regions of interest. ***P<0.0001 (unpaired Student's t test)

FIG. 2A-G illustrate: Representative images of periodontal probing depth measurement in an example method.

FIG. 2A illustrates Illustration of distal, lingual, and mesial probing locations in the porcine tooth.

FIG. 2B illustrates The photoacoustic image from a tooth with stains and a contrast agent with 680-nm excitation.

FIG. 2C illustrates The contrast agent (upper line) and stain (lower line) had a similar signal at 680 nm, but the signal of the stain decreased about 50% at 800 nm. This was used to discriminate between the two absorbers. Photoacoustic/ultrasound images before FIG. 2D and after FIG. 2E cuttlefish ink/cornstarch contrast agent treatment. The resulting blue color is a photoacoustic signal from stains on the teeth, and the resulting red color (lower portion of FIG. 2E is photoacoustic signal from a cuttlefish ink/cornstarch contrast agent. The (green) dashed line is the gingival margin, and the (yellow) vertical bar represents the probing depth.

FIG. 2F illustrates This sagittal view of a 3-dimensional scan on a tooth clearly illustrates both hard and soft dental tissues, and the photoacoustic signal of the contrast agent in the pocket started from the gingival margin and extended along the root direction analogous to a probing depth measurement. This sagittal view shows the tooth surface, gingival margin, gingiva, and bone; the probing depth is obvious when the tooth is viewed in a sagittal plane after contrast agent treatment.

FIG. 2G illustrates Photoacoustic/ultrasound images acquired before and after ink treatment, followed by multiple water rinses. The contrast agent remained in the pocket for five rinses (only three shown here for economy of space). The contrast agent was easily removed after brushing. The scale bars are 1 mm.

FIG. 3A-D illustrate: Periodontal probing depth measurements and comparison between methods.

FIG. 3A illustrates Representative images, including shallow (1.65 mm; i), intermediate (2.04 mm; ii), deep (4.45 mm; iii), and artificially deep (4.60 mm; iv) pockets obtained via photoacoustic imaging according to an example method. Comparisons of probing depth measured by periodontal probe and by imaging at FIG. 3B mesial, FIG. 3C lingual and buccal, and FIG. 3D distal locations of molars. The (red) circles and hollow squares are the results of natural and artificial pockets, respectively. The bars indicate the mean probing depth measured by photoacoustic imaging. The error bars represent the standard deviation of the depths measured by photoacoustic imaging.

FIG. 4A-D illustrate: Bland-Altman plots. Comparison of agreement between probing depths measured by photoacoustic imaging according to an example method and Williams probe at FIG. 4A mesial, FIG. 4B lingual and buccal, and FIG. 4C distal locations of molars. The (blue) circles and hollow squares are the results of natural and artificial pockets, respectively. The bias values were calculated by subtracting the depth measured by photoacoustic imaging by the depth measured with the Williams probe.

FIG. 4D The coefficient of variation for the five replicates in mesial, lingual/buccal, and distal locations were all <11% for all 13 teeth. The mean coefficient of variation values were approximately 6%.

FIG. 5A-B illustrate: Gingival thickness measurement.

FIG. 5A illustrates Representative ultrasound mode images showing the precise measurements obtained with a 40-MHz ultrasound transducer.

FIG. 5B illustrates Comparison of agreement between gingival thicknesses measured by ultrasound imaging and by needle 2 mm from the gingival margin. The difference was calculated by the thicknesses measured by ultrasound minus the value obtained with a needle.

FIG. 6A-C illustrate an exemplary product of the invention and an example method of use wherein a device embodied in a mouthpiece or mold

FIG. 6A contains light-emitting diode (LED) excitation and acoustic transducers disposed therein; and a dental patient, or an individual in need thereof (including humans or animals), wears the device during examination (the image showing insertion of the device into the mouth), or has the device placed upon the teeth (the dental arch to be monitored) after an oral rinse with a contrast agent FIG. 6B; in an alternative embodiment the device electronically transmits (optionally by wire connection or wirelessly (FIG. 6C)) the results to a remote receiver or record, e.g., a computer and/or a database, e.g., an electronic medical record or a computer (e.g., hard disk) or a cloud-based storage source.

FIG. 7 illustrate an example imaging setup according to another example method, in which a photoacoustic ultrasound (PA-US) transducer (II.) is connected to a stepper motor (III) for axial scanning and a sliding positioner (IV) for lateral control, and these components are attached to a larger frame for general positioning. A head immobilizer (I.) (cross-section shown) rests on a flat surface.

FIG. 8A-B illustrate ultrasound (US) images of ten teeth acquired from a human subject at 40 MHz and comparison to images at lower frequencies:

FIG. 8A illustrates Frontal and sagittal images of the bottom front incisors (24 and 25) at increasing US frequencies. Frontal images are inset within the magnified sagittal images of tooth 25 at the gingival margin. Scale bars are 1 mm and apply to all frequencies for the sagittal images.

FIG. 8B illustrates Frontal and sagittal cross-sections of teeth 7-10 and 22-27. The frontal and sagittal views are composites of planes from ten separate scans. The gingiva, gingival margin, and tooth are resolved in each sagittal image. The 3-mm scale bar is for sagittal images.

FIG. 9A-F illustrate photoacoustic-ultrasound images of the bottom front incisors (24, 25) before and after administration of an example contrast agent with demonstrated reproducibility:

FIG. 9A illustrates Frontal cross-section of teeth 24 and 25 with photoacoustic signal overlaid on ultrasound prior to administration of contrast agent.

FIG. 9B illustrates Sagittal cross-section of tooth 25, indicated by the dashed (yellow) line in Panel A, with tooth, gingival margin, and gingival thickness labelled.

FIG. 9C illustrates Frontal cross-section of teeth 24 and 25 with photoacoustic signal overlaid on ultrasound after administration of contrast agent.

FIG. 9D illustrates The sagittal cross-section of tooth 25 (dashed (yellow) line in FIG. 9C shows the soft-hard tissue interface and pocket revealed by the photoacoustic contrast agent below the gingival margin. (FIG. 9E, FIG. 9F) Reproducibility of measurements for representative sagittal planes at mesio-buccal, central, and distobuccal probing locations across five replicates in tooth 25; labeling and imaging procedures were performed independently from beginning to end. Each PA-US image in FIG. 9F corresponds to the mesio-buccal probing depth measurement (squares) for its given replicate number on the x-axis in FIG. 9E. Light yellow lines show the pocket measurements. White: ultrasound, red: photoacoustic.

FIG. 10A-E illustrate mapping the full width and contours of the periodontal pocket in an example method.

FIG. 10A illustrates US frontal image of tooth 25 prior to administration of contrast agent.

FIG. 10B illustrates PA-US frontal image of the same tooth following contrast agent with the subgingival region of interest highlighted by a black box, prior to image processing.

FIG. 10C illustrates A sagittal view of the tooth at the (yellow) vertical dashed line from FIG. 10A and FIG. 10B used for image processing. The (yellow) vertical line segment represents the pocket measurement and the (green) vertical arrow indicates the distance from the crown of tooth 25 to the gingival margin; these two metrics were used to determine the top and bottom of the pocket for that sagittal plane.

FIG. 10D illustrates Pocket measurements for every sagittal plane across the full buccal width of the tooth—a total of 40 planes were measured. The x-axis represents each sagittal slice across the width of the tooth. The shaded region represents the geometry of the pocket and the white region represents the normal area of attachment between tooth and epithelia. Error bars show the standard error of the mean for five replicates (n=5).

FIG. 10E illustrates The final mapping of the pocket generated by an example photoacoustic method. It was generated by taking the measurements for each sagittal slice from FIG. 10D and overlaying them on the ultrasound image. This step removes the non-specific photoacoustic signal from FIG. 10B.

FIG. 11A-B illustrate removal of contrast agent following oral rinsing with water and teeth brushing:

FIG. 11A illustrates Representative PA-US images show the reduction in photoacoustic signal/pocket measurement following oral rinsing with water for 15 s, 30 s, and brushing. (Yellow) line segments indicate the pocket measurements.

FIG. 11B illustrates Fraction of original measurement following washing steps for five representative sagittal planes taken from teeth 24 and 25. The dotted line indicates the original measurement and error bars show SEM (n=5). Asterisks denote significant difference from original measurement (unpaired t-test), ** (P≤0.01), *** (P≤0.001).

FIG. 12 illustrates photoacoustic-ultrasound images (B-Mode) of gingival blood oxygen saturation for the lower incisors of a rabbit according to another example method. The rabbit inhaled air across a range of oxygen concentrations: 100%, 30%, 21%, 15%, 10%, and 5%.

FIG. 13 graphically illustrates a correlation between pulse oximetry and gingival photoacoustic measurements of blood oxygen saturation across the range of oxygen tensions shown in FIG. 12. The animal inhaled different oxygen tensions and the oxygenation was measured with the two different approaches.

FIG. 14A-B illustrate real-time decline of blood oxygen saturation in the rabbit gingiva while inhaling 100% nitrogen, with FIG. 14A graphically illustrating the data of the image of FIG. 14B.

FIG. 15 graphically illustrates real-time monitoring of blood oxygen saturation in the rabbit gingiva while alternating between ambient air and 100% nitrogen. Photoacoustic and pulse oximetry methods are both shown.

Like reference symbols in the various drawings indicate like elements.

### DETAILED DESCRIPTION

In alternative embodiments, provided are methods, products of manufacture, and kits effective for measuring and high-spatial resolution imaging of periodontal tissues, including periodontal pockets, including high-spatial resolution imaging to image and measure probing depths of periodontal pockets. In alternative embodiments, provided are methods and products of manufacture using photoacoustic imaging for probing depth measurements with applications to the dental field, including but not limited to use as tools for automated dental (e.g., periodontal) examinations or noninvasive examinations.

In alternative embodiments, example methods and products of manufacture and kits comprise use a combination of ultrasound and optical imaging referred to as photoacoustic (PA) imaging, in tandem with an oral rinse, e.g., a food based imaging agent, e.g., an imaging agent based on food-grade squid ink and cornstarch, or equivalents, that the individual in need thereof, e.g., a dental patient, can use briefly before imaging. In alternative embodiments, the rinse comprising the imaging agent (e.g., comprising food-grade squid ink, or melanin) increases the amount of photoacoustic signal in the pocket depth because it contains strong optical absorbers such as melanin, e.g., it contains strong optical absorbers from the squid ink (melanin).

An example method uses down to 10 microliters of imaging (contrast) agent per pocket, though this amount can be greater or smaller. In a particular example described below (Example 1), the imaging agent includes melanin nanoparticles. Alternative or additional imaging agents include, but are not limited to an FDA approved dye such as methylene blue or indocyanine green, activated charcoal, so long as the material absorb light and be safe for the animal or human being tested. The taste of the imaging agent can also be configured to be more acceptable by the subject for use, as will be appreciated by those of ordinary skill in the art.

In alternative embodiments, any one or several contrast agent(s) that can image below the gum line (e.g., into the periodontal pocket), e.g., that can image a bacterial infection or inflammation below the gum line, can be used. In alternative embodiments, inflammation in the gums or below the gum line (e.g., into the periodontal pocket) is imaged by photoacoustic spectroscopy, e.g., by measuring hemoglobin/deoxyhemoglobin levels or concentrations in the gum tissue to quantify the inflammation.

In alternative embodiments, products of manufacture as provided herein can make pocket depth measurements automatically, e.g., to allow dentists or periodontists to see more patients or employ fewer hygienists.

In alternative embodiments, products of manufacture as provided herein can observe or measure soft tissue contrast, e.g., gums, the periodontium, or the oral mucosa can be imaged and/or measured. In comparison, X-ray and computer tomography (CT) imaging can only image hard tissues, e.g., bone.

In alternative embodiments, products of manufacture as provided herein can image bacterial infection, e.g., including the resulting inflammation, which can be difficult to identify during a dental exam.

In alternative embodiments, products of manufacture as provided herein are used with an oral rinse that absorbs light and converts that to acoustic waves. This rinse penetrates periodontal pockets and enhances contrast to measure pocket depths. This device works by using both ultrasound and photoacoustic signal. The ultrasound is specific to bones and some gum contrast, while the photoacoustic signal offers specific information on plaque, pocket depths, and/or bacterial infection.

In alternative embodiments, the term “photoacoustic detectable signal” as used herein refers to a signal derived from the contrasting agent absorbing light energy and converting it to thermal energy that generates the photoacoustic signal. The photoacoustic detectable signal is detectable and distinguishable from other background photoacoustic signals that are generated from the subject or sample. In other words, there is a measurable and statistically significant difference (e.g., a statistically significant difference is enough of a difference to distinguish among the photoacoustic detectable signal and the background, such as about 0.1%, 1%, 3%, 5%, 10%, 15%, 20%, 25%, 30%, or 40% or more difference between the photoacoustic detectable signal and the background) between photoacoustic detectable signal and the background. Standards and/or calibration curves can be used to determine the relative intensity of the photoacoustic detectable signal and/or the background.

In alternative embodiments, the term “photoacoustic imaging” as used herein refers to signal generation caused by a light pulse, absorption, and expansion of a contrast agent, followed by acoustic detection, where the contrasting agent absorbs the light energy and converts it to thermal energy that generates the photoacoustic signal. In alternative embodiments “photoacoustic imaging” also can refer to converting the detectable signal into a data form that may then be transformed into an image of the signal within the cell/s, tissue/s, or living animal or human, said image being visible and interpretable by an operator.

In alternative embodiments, the acoustic signal(s) are detected and quantified in real time using an appropriate detection system, i.e., any detection system known in the art. For example, two instruments that can be used quantifying acoustic signal are the NEXUS128™ (Endra Life Sciences, Ann Arbor, MI), and the VEVO™ 2100 (Fujifilm VisualSonics, Inc., Toronto, Canada). Others can be used and purchased from manufacturers such as but not limited to iThera. The units of acoustic signal can vary and include echogenicity units (EU) or mean grey scale. Input units include dB and frequency (MHz). Maximum intensity persistence imaging can also be used and is described by Pysz et al. in Investigative Radiology (2011) 46:187-195. In alternative embodiments, other detection strategies, including capacitive micromachined ultrasonic transducers (CMUT) arrays, are also used to detect the acoustic signal.

In alternative embodiments, provided are kits that comprise products of manufacture and dental devices comprising photoacoustic probes and directions (written instructions for their use). These components can be tailored to the particular disease, biological event, or the like, being studied, imaged, and/or treated (e.g., cancer, cancerous, or precancerous cells). The kit can further include appropriate buffers and reagents known in the art for administering various combinations of the components as provided herein.

In alternative embodiments, acoustic energy is detected and quantified in real time using an appropriate detection system. The acoustic signal can be produced by one or more photoacoustic probes as provided herein. In alternative embodiments, exemplary acoustic energy detection systems are as described in: J. Biomedical Optics (2006) 11, p024015; Optics Letts. 30:507-509, each of which are included herein by reference. In an embodiment, the acoustic energy detection system includes a 5 MHz focused transducer (25.5 mm focal length, 4 MHz bandwidth, F number of 2.0, depth of focus of 6.5 mm, lateral resolution of 600 μm, and axial resolution of 380 μm. A309S-SU-F-24.5-MM-PTF™, Panametrics), which can be used to acquire both pulse-echo and photoacoustic images. In addition, high resolution ultrasound images can also be simultaneously acquired using a 25 MHz focused transducer (27 mm focal length, 12 MHz bandwidth, F number of 4.2, depth of focus of 7.5 mm, lateral resolution of 250 μm, and axial resolution of 124 μm. V324-SU-25.5-MMTM Panametrics). Other detection strategies including capacitive micromachined ultrasonic transducers (CMUT) arrays can also be used to detect the acoustic signal.

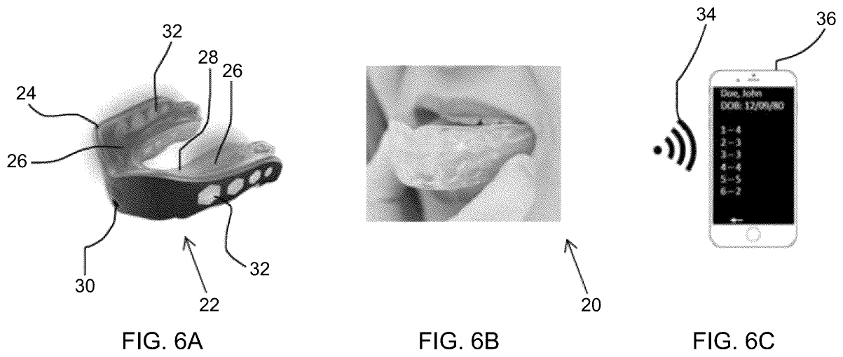

Referring now to the drawings, FIG. 6 shows components of an example system 20 for imaging a periodontal tissue or a periodontium of an animal, including a human. The periodontal tissue may, for instance, include the oral mucosa or the gingiva.

In alternative embodiments, example methods and products of manufacture and kits use a mouthpiece or mold (“mouthpiece” herein) 22 that is capable of partially or fully receiving (e.g., fitting over or covering) one or several teeth, or all teeth in an arch, and optionally also fitting over or covering (e.g., at least part of) the periodontium or gingiva. The arch can be, for example, a maxillary arch, a mandibular arch, or both. The example mouthpiece 22 is sized and shaped for fitting over several teeth in an arch, optionally also fitting over or covering some gingiva or periodontium, although this is not necessary in all embodiments.

In alternative embodiments, the example mouthpiece 22 has an outer body 24 that is similar to that of a conventional mouthpiece such as a 6-9 cm wide and 2-4 cm high. The outer body 24 can be molded or otherwise formed from conventional mouthpiece or mold materials, such as but not limited to acrylic, vinyl, or poly(methyl methacrylate) (PMMA) or any appropriate polymer. In alternative embodiments, the mouthpiece, or any subsection, subpiece or all structural components of a product of manufacture or dental device as provided herein can be 3D printed.

In alternative embodiments, disposed within or on the outer body 24 are one or more light generators 26, such as but not limited to light-emitting diodes (LEDs). As shown in FIG. 6A, the light generators 26 are disposed and configured such that light signals, such as light pulses, can emanate from the light generators and penetrate the contrasting agent when the mouthpiece 22 is fitted over the one or more teeth. For example, the light generators 26 are disposed over a portion of the outer body 24, e.g., received within openings in a gum-facing surface 28 of the outer body, so that teeth and gingiva positioned to abut the gum-facing surface receive the generated light pulses. The light generators 26 may be driven by a suitable driving circuit (not shown) coupled thereto, which may be disposed within the outer body 24 or external to the outer body, such as via line 30. The light generators 26 may be configured or operated to generate light pulses having a frequency of about 5 to 5,000 pulses per second.

In alternative embodiments, further disposed within or on the outer body 24 are one or more transducers 32, e.g., acoustic transducers, for receiving an acoustic (photoacoustic) signal from the contrast agent and generating a data signal. Example transducers 32 include, but are not limited to, ultrasonic transducers, such as piezoelectric transducers, patterned silicone elastomers and the like. In an example embodiment, the transducers 32 are disposed within openings in the outer body 32 and arranged therein to generally align with target areas to be imaged, such as gingiva at one or more teeth. In example embodiments, an acoustic coupling medium or gel, or equivalent, can be provided to transmit acoustic waves from an oral mucosa, gum, tooth, or periodontal pocket to the transducers 32. Receiving circuitry (not shown) coupled to the transducers 32 may be disposed within the outer body 24 or external to the outer body, such as via line 30.

The mouthpiece 20 may, but need not, also include one or more wireless transmitters 34 and/or receivers (not shown) for transmitting data signals generated by the transducers 32 and/or for receiving control signals to operate the light generators 26. In the example mouthpiece shown in FIG. 6B, the mouthpiece 20 includes a wireless transmitter (e.g. a radio frequency (RF) transmitter 34 of any suitable type, as will be appreciated by those of ordinary skill in the art) transmitting some or all of the generated data signals to a detection system, such as may be embodied in a processing device 36, e.g., portable communication device (such as but not limited to a smartphone, executing a suitable application (app)). Other example processing devices are disclosed herein. Alternatively or additionally, the mouthpiece 20 may include an internal data memory storage (not shown) for storing data signals generated by the transducers 32, which then (e.g., after removal of the mouthpiece from the patient's mouth) can be uploaded (either via signal wires or wirelessly) to a processing device such as those described herein.

The processing device can include a processor (which can include one or several processors), a memory (or several memories), an (optional) database(s), wired and/or wireless interface(s), and suitable computer-readable instructions stored on a non-transitory medium or media for causing the processor to perform example methods for receiving and processing image data signals to generate an image as disclosed herein. Alternatively or additionally, the processing device 36 may be configured to receive data signals (e.g., locally), and store and/or forward (wired or wirelessly, via a network, the Internet, etc.) the received data signals to an operatively linked remote receiver, such as another processing device. Particular, non-limiting example processing devices 36 for processing generated photoacoustic data signals are disclosed herein. Alternatively, receiving of data signals and/or generation of control signals can be provided via a wired connection such as through line 30 or elsewhere. Example processing methods can be performed by multiple processing devices operating example method steps in series or in parallel, and such multiple processing devices can be considered a “processing device” as used herein.

The received data signals, providing ultrasound and/or optical imaging data, are preferably capable of being converted to: an image of the periodontium or periodontal tissue, or an image of a periodontal pocket, or a periodontal probing depth measurement. In example embodiments, the processing device 36 is configured to convert the received data signals to: an image of the periodontium or periodontal tissue, or an image of a periodontal pocket, or a periodontal probing depth measurement. Example methods for converting the received data signals to images are disclosed herein. Generated images can be displayed as an interactive or non-interactive image, printed (hard printing or into a different format), stored in a database or other non-transitory memory, transmitted to another device, etc.

A suitable power supply (not shown), such as but not limited to a battery as will be appreciated by those of ordinary skill in the art, may be provided within the outer body 24 for providing power to one or more of the light generators 26, transducers 32, transmitters 34, or receivers if power for any or all of these is not provided via wired connection. Alternatively, power (and signals) for one or more of these may be provided via line 30 or other line.

An applicator (not shown) may be provided for providing (e.g., applying) the imaging or contrasting agent to the patient's teeth and/or gingiva or periodontium. The applicator may be, for instance, a container (e.g., a bottle) holding the imaging agent, a syringe, a pipette, a tray (e.g., fitted or formed to fit the dental arch or the teeth to be measured), or any injection device. If the imaging or contrasting agent is provided, for instance, in a mouth rinse, the applicator may simply be a container holding the mouth rinse (and optionally a dispenser).

In an example operation as shown in FIG. 6B, the individual in need thereof, e.g., a dental patient, holds in their mouth (or have placed or held in the mouth on the teeth) the mouthpiece 22, e.g., briefly, for, e.g., rapid (e.g., less than 5 second) measurements of pocket depths, optionally also giving/reading detailed information on inflammation of the gingiva. The contrasting (imaging) agent can applied via any means, e.g., tray, syringe, pipette, etc. The light generators 26 produce one or more light pulses over the measurement duration, and the optical absorber within the imaging agent produces an acoustic signal, which then is read by one or more of the transducers 32 to provide one or more data signals. The data signals are transmitted (and optionally stored, then transmitted) to the (preferably external) processing device 36 for processing the data signals to provide one or more images, such as by using methods explained further below.

Particular embodiments of the invention will be further described with reference to the examples described herein; however, it is to be understood that the invention is not limited to such examples.

### EXAMPLES

### Example 1: Exemplary Method and Products of Manufacture

This example demonstrates that exemplary methods and products of manufacture as provided herein are effective for measuring and high-spatial resolution imaging of periodontal tissues, including periodontal pockets, including high-spatial resolution imaging to image and measure probing depths of periodontal pockets. Photoacoustic (PA) imaging is used in tandem with an imaging agent, e.g., a food-grade oral rinse, to estimate probing depths in a porcine model, and the results were compared with a gold standard periodontal probe. The results demonstrate that photoacoustic imaging can be a complementary tool for the dental community to image pockets and measure probe depth noninvasively.

Photoacoustic ultrasound is used for high-spatial resolution imaging of probing depths. Photoacoustic imaging is a hybrid imaging modality that combines visible and near infrared excitation with acoustic detection (Wang and Hu 2012). It extends the utility of ultrasound by enabling contrast based on optical absorption (Beard 2011). Traditional ultrasound operates under the principle of “sound in, sound out”, but photoacoustic imaging shifts this concept to “light in, sound out” (Zackrisson et al. 2014).

Here, a near infrared laser excites a light-absorbing target. The target then undergoes spatially confined heating followed by thermoelastic expansion. This generates wideband acoustic waves that can be detected with high frequency ultrasound transducers for image generation. Photoacoustic imaging has been largely investigated for monitoring disease states, therapies, and image-guided surgeries (Mallidi et al. 2011; Emelianov et al. 2009). Though photoacoustic-ultrasound (PA-US) signal can be limited by tissue penetration, it has two significant advantages over radiography, the most common dental imaging modality (Vandenberghe et al. 2010): (1) it can image soft and hard tissue alike; and (2) it does not use ionizing radiation.

Specific contrast from dental pockets was achieved with food-grade cuttlefish ink as a contrast medium. Here, 39 porcine teeth (12 teeth with artificially deeper pockets) were treated with the contrast agent, and the probing depths were measured with novel photoacoustic imaging and a Williams periodontal probe. There were statistically significant differences between the 2 measurement approaches for distal, lingual, and buccal sites but not mesial. Bland-Altman analysis revealed that all bias values were <±0.25 mm, and the coefficients of variation for 5 replicates were <11%. The photoacoustic imaging approach also offered 0.01-mm precision and could cover the entire pocket, as opposed to the probe-based approach, which is limited to only a few sites. This demonstrates the use of photoacoustic imaging for probing depth measurements with applications to the dental field, including use as tools for automated dental examinations or noninvasive examinations.

### Materials and Methods

### Reagents

Cuttlefish ink A was obtained from Conservas de Cambados, and ink B was obtained from Nortindal. Both contained cuttlefish ink, water, salt, and sodium carboxymethyl cellulose as a thickener. Cornstarch was purchased from a local food store. Porcine heads were purchased from a local butcher. Agarose was purchased from Life Technologies. India ink solution 0.2% in phosphate-buffered saline (PBS) buffer was purchased from Thermo Fisher Scientific. Intralipid 20% and PBS tablets were purchased from Sigma-Aldrich. Polyethylene tubing (outside diameter: 1.27 mm, inside diameter: 0.85 mm) was purchased from Harvard Apparatus.

### Equipment

Transmission electron microscopy (TEM) images were performed with a JEOL JEM-1200-EX II™ operating at 80 kV. The absorbance spectra were measured with a SPECTRAMAX M5™ spectrophotometer. The hydrodynamic radius was measured with a ZETASIZER™ from Malvern via dynamic light scattering. The photoacoustic images were performed with a Vevo LAZR™ imaging system (Visualsonics) equipped with a 21 or 40 MHZ-centered transducer as described previously (Ho et al. 2015; Wang et al. 2016).

### Preparation of Cuttlefish Ink Derivatives

To measure absorbance and photoacoustic spectra, 50% ink A (weight per volume) and 10% ink B (weight per volume) were prepared with 0.1M PBS solution, sonicated for 1 h, and further diluted. Ink A was diluted 50-fold and ink B, 10-fold, with 0.1M PBS solution to make a 1% solution. These samples were used for absorbance measurements; 1% solutions were made for photoacoustic spectral analysis. Corn starch (0.04 g) and ink A (0.04, 0.2, and 0.4 mL) were mixed in 0.1M PBS and boiled to prepare 2 mL of cornstarch-enhanced contrast agent. The pH of freshly prepared contrast agent with 2% cornstarch and 5% cuttlefish ink was 7.4. This was adjusted to pH 6.2, 6.6, 7.0, and 7.8 with 6N HCL and/or NaOH. Samples were placed in tubing for photoacoustic imaging.

### Preparation of Cuttlefish Melanin Nanoparticles for TEM Imaging

A stock solution of ink A was diluted into 1% in Millipore water, sonicated for 1 h, and centrifuged at 5,000 g for 15 min (Chen et al. 2009). The supernatant was removed, and the pellet was resuspended in MILLIPORE™ water. This was repeated 6 times, and the resulting pellet was suspended in pure ethanol for TEM imaging or PBS for dynamic light scattering.

### Preparation of Tissue-Mimicking Phantom

The tissue-mimicking phantom was prepared with 0.5% (weight per volume [w/v]) ultrapure agarose solution, approximately 50% (volume per volume) India ink as the absorber, and 0.5% (w/v) INTRALIPID™ 20% as the scatter (Hanli et al. 1995; Nagarajan and Zhang 2011). These were prepared in 1% (w/v) ultrapure agarose. A custom phantom was used to measure penetration depth (Arconada-Alvarez et al. 2017).

### Preparation of Porcine Jaw Samples

Frozen porcine heads were sliced sagittally with a band saw. The lower jaw was removed with a handsaw; soft tissues surrounding the jaw were dissected with a scalpel. Artificial deeper pockets were created with scalpels (Dynarex) to simulate periodontal lesions. The scalpel was applied parallel to the tooth with intrasulcular incisions until the scalpel contacted the bone (Weidmann et al. 2014). The contrast agent was pipetted onto the gingival line, and excess was rinsed with a syringe and 5 mL of deionized water. The jaw was immobilized in water for ultrasound coupling and imaged with a Vevo LAZR™.

To test the signal stability, 1 molar of the mandible was rinsed 5 times with water after labeling. The jaw was imaged after each rinse to observe the photoacoustic signal. The tooth was then brushed for about 1 min without toothpaste after the final rinse. This study was expanded to 13 molars. For each, the tooth was labeled, imaged, and brushed 5 times to calculate a coefficient of variation for the probing depth.

### Periodontal Probing Depth Measurements

The probing depth was measured with a Williams probe following the direction of the tooth root (Mayfield et al. 1996) before photoacoustic imaging. The same examiner measured the periodontal probing depths with photoacoustic imaging and a Williams periodontal probe (Listgarten 1980). The Williams probe was marked at 1, 2, 3, 5, 7, 9, and 10 mm. In the maxilla, the probing depths were recorded at 4 sites per tooth: the mesial and distal sites of the tooth as well as 2 buccal locations below the anterior and posterior cusps of maxillary molar and fourth premolar. In the mandible, probing depths were recorded on the mesial and distal ends of the tooth as well as at 2 lingual locations below the anterior and posterior cusps of the mandibular molar, as described in more detail below.

### Gingival Thickness Measurements

The gingival thickness values were measured with ultrasound imaging and compared with values collected with a needle and dental gauge analogous to methods with the UNC-15 and No. 25 K-file instruments (Vandana and Savitha 2005; Slak et al. 2015). Here, for each tooth, the measurement points were 2 mm below the gingival margin. A 28-gauge needle was then inserted perpendicularly into gingiva until it made contact with the tooth. A line was then drawn on the needle where it made contact with the gingiva. After removal of the needle, the distance between the marked location and the tip of the needle was measured with dental gauge (0.1-mm precision).

### Imaging Procedures

Typical imaging conditions included 100% laser energy; typical gains were 20 dB for photoacoustic and 10 dB for ultra-sound. Photoacoustic spectra were collected from 680 to 970 nm. Porcine jaws were aligned parallel to the 40-MHz transducer and scanned from the crown to the root. The 3-dimensional scans were performed by oscillating between 680- and 800-nm excitation. All 3-dimensional images were processed as a maximum intensity projection.

### Image Processing

Photoacoustic data were analyzed with IMAGEJ 1.48™ (Abramoff et al. 2004). The intensity of each tube was measured in 8 regions of interest for statistical analysis. To discriminate the photoacoustic signal from stains and contrast agent, data at both was collected 680 and 800 nm. The image at 680 nm was subtracted from the one at 800 nm excitation. The resulting pixels were coded blue. These blue pixels were overlaid on the original image created with 680-nm excitation that had already been coded red. Only 680-nm excitation was used to image stain in the absence of contrast agent. The periodontal probing depths were measured on the sagittal view of 3-dimensional images with the VEVO LAB™ software.

### Statistical Treatment

The mean, standard deviation, Bland-Altman plots, and coefficient of variation (CV) were based on GRAPHPAD PRISM 5™ (Bland and Altman 1986). All error bars represent the standard deviation. Here, P<0.05 was considered significant.

### Results

### Characterization of Cuttlefish Ink Derivatives

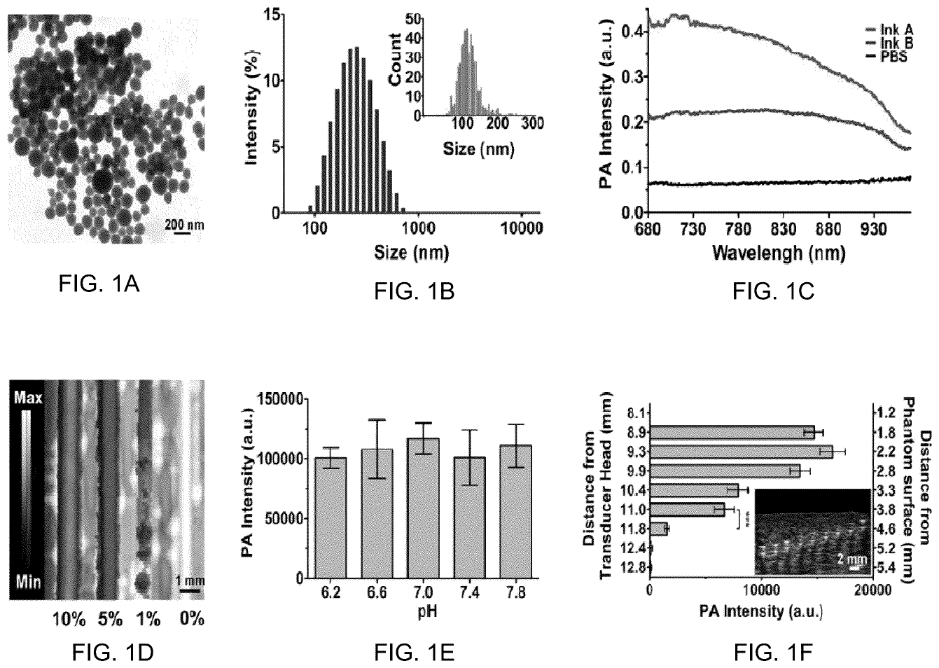

TEM (FIG. 1A) showed that the cuttlefish ink contained spherical melanin nanoparticles (Ju et al. 2013); the measurement of 500 nanoparticles via TEM indicated a mean size of 125.1 nm (FIG. 1B). Dynamic light scattering showed that the mean size was 266.3 nm with a polydispersity index of 0.116 (i.e., a measure of size uniformity; <0.3 indicates uniformity). Both inks had a broad photoacoustic spectrum from 680 to 970 nm; ink A had 2-fold-stronger signal intensity than ink B at 680 nm (both at 1%; FIG. 1C). Thus, ink A was used for all subsequent experiments.

FIG. 1D shows the photoacoustic (red) and ultrasound (black and white) mode images of the ink/cornstarch contrast agent at different concentrations. The photoacoustic signal increased proportionally to ink concentration (R²=0.96); 2% cornstarch alone had no photoacoustic signal. The effect of pH on photoacoustic signal was also evaluated, but no significant change from pH 6.2 to 7.8 was observed (FIG. 1E). The photoacoustic signal of the contrast agent as a function of depth in tissue phantom was quantified (FIG. 1F) and showed that 1-cm imaging was routine.

### Periodontal Labeling

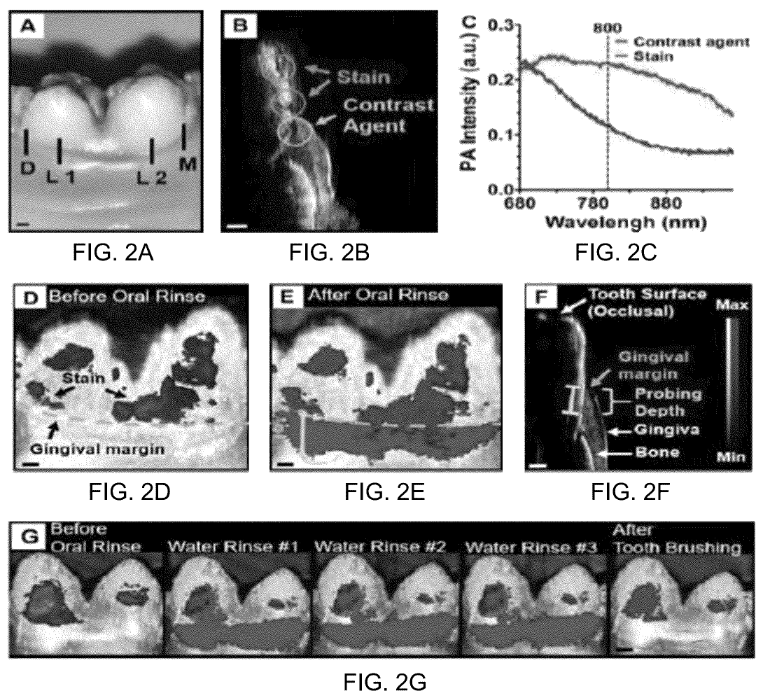

Next, the probing depth was measured with imaging and a Williams probe (FIG. 2A) at mesial, distal, lingual, and buccal locations. Imaging was complicated by endogenous stains on the porcine teeth (FIG. 2B). Fortunately, the contrast agents had similar signal at 800 and 680 nm, but the signal of the stains decreased about 50% at 800 nm relative to 680 nm (FIG. 2C). Thus, photoacoustic spectroscopy was used to discriminate between stained teeth (FIG. 2D) and the signal of the contrast agent (FIG. 2E).

FIG. 2G shows combined ultrasound and photoacoustic images in a sagittal view of the tooth. Hard and soft dental tissues were obvious. The photoacoustic signal in the pocket started from the gingival margin and extended along the root. The stability with additional water rinses was also evaluated, but the signal did not decrease (<0.2% after 5 washes). This was repeated at the mesial, lingual, and distal locations, including up to 5 rinse cycles, but no significant decrease was seen (P>0.05). The contrast agent was easily removed via teeth brushing.

### Periodontal Probing Depth Measurements

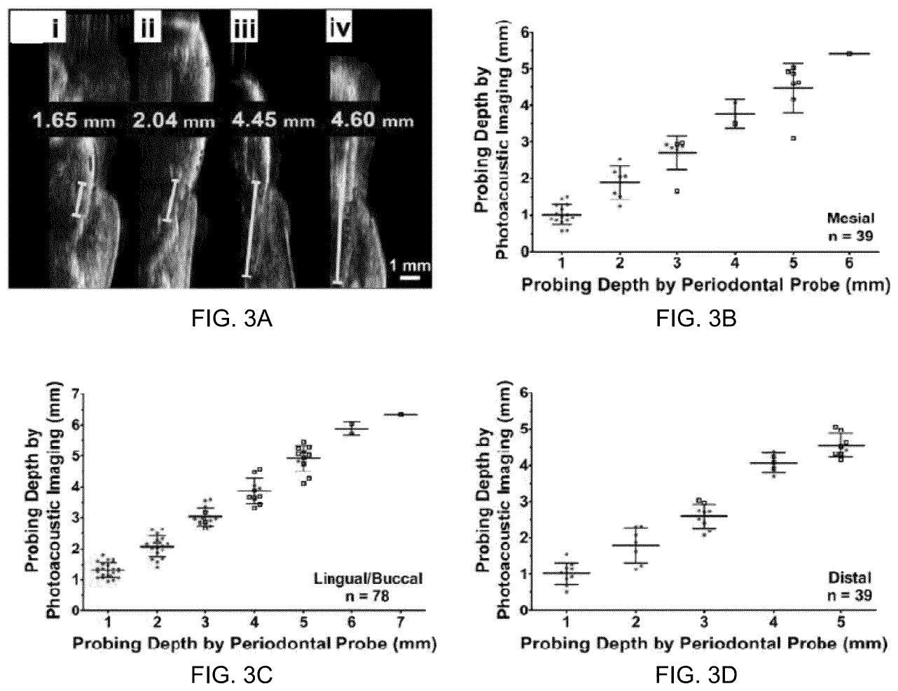

FIG. 3A shows shallow, intermediate, and deep pockets and artificially created deep pockets. The artificially deep pockets were created because swine have shallow pockets, but evaluating the technique in deep pockets was a key goal. FIGS. 3B-D compares the periodontal probing depths measured by photoacoustic imaging with depths measured with a probe at mesial (n=39), lingual and buccal (n=78), and distal (n=39) locations. The lingual and buccal groups were combined because of their similar geometry.

Photoacoustic data was compared with Williams probe data via a paired t test. There was no significant difference for the mesial data, but the distal and lingual/buccal groups were significantly different (P<0.05). The distal and mesial pockets were underestimated by photoacoustics, but the lingual and buccal sites were overestimated with photoacoustics relative to periodontal probe. The combined lingual/buccal groups were also divided into lingual only and buccal only. These also showed a significant difference (P<0.05). Thus, these data were further quantified via Bland-Altman analysis.

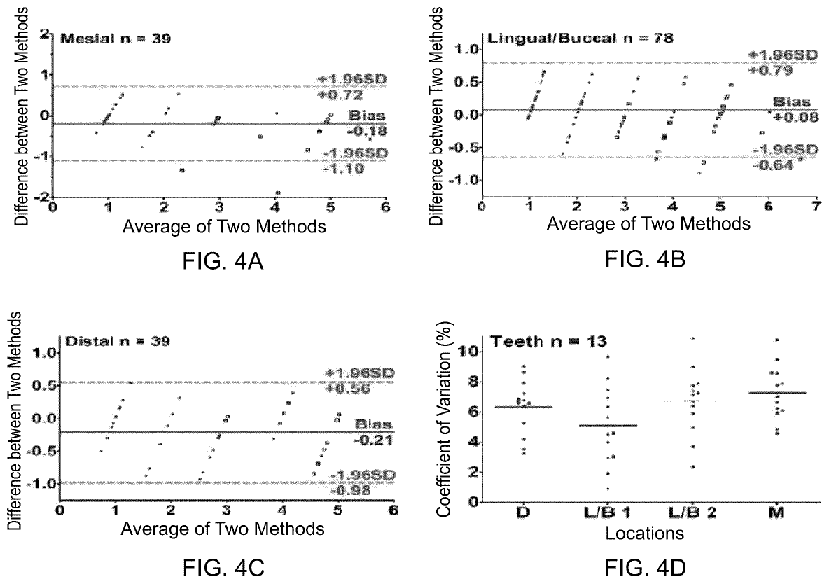

Bland-Altman plots show that 95% of the samples fell within 1.96 standard deviations of the differences between the 2 methods (95% confidence interval [95% CI]) at mesial, lingual and buccal, and distal locations (FIG. 4). Small bias values of −0.18, 0.08, and −0.21 mm were identified at mesial, lingual and buccal, and distal locations, respectively; the 95% CIs are plotted as well and are all <1.0 mm, except at mesial locations (FIG. 4). Thus, the photoacoustic measurements were slightly lower, higher, and lower at mesial, lingual and buccal, and distal locations, respectively. Bland-Altman analysis was repeated separately for lingual and buccal sites. Buccal (n=56) sites had a bias of 0.05 mm (95% CI, 0.81 to −0.72 mm); lingual (n=22) sites had a bias of 0.20 mm (95% CI, 0.84 to −0.44 mm).

Additional experiments were conducted to more carefully evaluate consistency of probing (FIG. 4D). 13 teeth were studied over 5 replicates. At each replicate, the mesial, distal, and lingual/buccal locations were measured. The CV for the 5 replicates were all <11%. The raw values for the CV are plotted in FIG. 4D for all 13 teeth. The mean CV values were ˜6%.

### Gingival Thickness Measurement

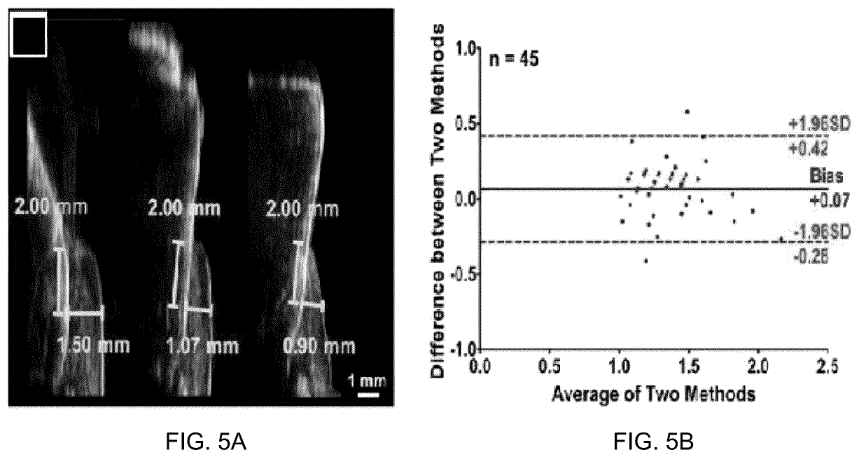

FIG. 5A shows that the gingival thickness can also be precisely measured via the ultrasound-mode data with a high-frequency ultrasound transducer (40 MHz). The gingival thickness measured with ultrasound imaging and with a needle for 45 teeth is 1.40±0.25 mm and 1.33±0.28 mm, respectively. The Bland-Altman plot (FIG. 5B) shows that the bias was 0.07 mm, indicating that needle measurements gave slightly lower values.

### Discussion

These experiments demonstrate the use of photoacoustic imaging with the melanin nanoparticles in cuttlefish ink as a contrast agent for noninvasive measurements of probing depths. Melanin has broad optical absorption for photoacoustic imaging (Viator et al. 2004). It is a common foodstuff with no safety concerns (Chaitanya 2014). The pH of saliva can range from 6.2 to 7.4 (Schipper et al. 2007), but this contrast agent has good signal stability regardless of pH (FIG. 1C).

Porcine teeth often have yellow-brown stains with background photoacoustic signal, but this could be spectrally discriminated from the squid ink contrast agent (FIG. 2B). Spectral imaging may have utility in further applications, including the use of multiple contrast agents concurrently for applications in mineralization or biofilm characterization.

One other issue with porcine models is the small probing depths relative to human subjects (2 to 3 mm, healthy; 4 to 5 mm with gingivitis; Wang et al. 2007). Most pockets were <3 mm, and only approximately 10% of teeth had pockets >3 mm; thus, we created 12 deeper pockets were created and repeated the imaging and depth measurements were repeated. These are shown as squares in FIG. 3. These artificial pockets also correlated to the Williams probe. The data points were included in the Bland-Altman analysis in FIG. 4 but only minor changes (bias of −0.04 to −0.18 mm for mesial, 0.17 to 0.08 mm for lingual/buccal, and −0.20 to −21 mm for distal locations) were noted.

Despite these challenges, the example photoacoustic imaging methods offer much more precise and continuous data on probing depths. In the clinic, probing depths are recorded at only 6 sites per tooth.

FIG. 3B-D show that this imaging approach can measure the probing depths at all points along the tooth. This can markedly minimize sampling error, and it might be able to detect isolated deeper pockets that may be indicative of vertical root fractures. This imaging approach also eliminates variation in the probe insertion point and angle and could eliminate the error results from the presence of calculus.

The spatial resolution of the 40-MHz transducer is 100 μm by ultrasound and approximately 300 μm in photoacoustic mode. Thus, this approach offers more precision among different depths as opposed to the Williams probe, which rounds to the nearest millimeter. This may have utility in monitoring/predicting therapy where small changes can have significant implications (Fiorellini 2016). The photoacoustic approach had a positive bias in lingual and buccal locations perhaps because they were more easily aligned beneath the transducer (FIG. 4); mesial and distal sites are slightly more difficult to access with the transducer.

FIG. 5B shows that the thicknesses measured on ultrasound mode images were 0.07 mm larger than invasive examinations. Gold standard methods include the UNC-15 and No. 25 K-file instruments (Vandana and Savitha 2005; Slak et al. 2015), but these are quite painful and require anesthesia. Other studies have shown that the mean thicknesses measured with A-line ultrasound were slightly higher than the invasive method perhaps because of the pressure placed on the gingiva during a physical examination (Vandana and Savitha 2005; Slak et al. 2015). The experimental results disclosed herein have certain limitations. As one example, the optical excitation and acoustic pressure waves can be absorbed and scattered by bone. Thus, an example technique might have limited utility with infra-bony pockets as well as with interproximal pockets. Further, restorations might generate background interfering photoacoustic signal, but this could be gated out spectrally as with the aforementioned stains (Balderas-López et al. 1999). Still further, pressure of crevicular fluid in the pockets has been shown to cause incomplete penetration of local irrigations into the full depth of pockets (Fjærtoft et al. 1992). This might limit the depth with which the squid ink-based contrast could enter the pockets. Additionally, the porcine model had relatively shallow pockets that might not accurately represent human disease. While artificially deeper pockets were created, these may not truly mimic human disease.

The experiments disclosed above demonstrate that probing depths could be measured with photoacoustic imaging. Values that were achieved with this novel technique agreed nicely with the gold standard periodontal probe approach but were more precise, offered higher resolution images, and covered all areas of the tooth. The gingival thickness could also be easily measured.

It is also contemplated to use models of periodontal disease as well as automated algorithms to collect and process the data. Further, it is contemplated to use LED-based systems for reducing cost and complexity.

### Example 2: Exemplary Method and Products of Manufacture

In an example method according to another embodiment, photoacoustic-ultrasound was used to image both the full depths and geometries of pockets in healthy human adults for non-invasive monitoring of gingival health after local irrigation of the pocket with contrast media.

### Reagents and Equipment

Cuttlefish ink was purchased from Conservas de Cambados and contained ink, water, salt, and sodium carboxymethyl cellulose. Phosphate-buffered saline tablets were purchased from Sigma-Aldrich. Ultrasound gel was obtained from Next Medical Products. PA-US images were taken with a laser-integrated high frequency ultrasound system from VISUALSONICS™ (Vevo LAZR). A medical head immobilizer was purchased from DEALMED™ Disposable dental cheek retractors were obtained from URL DENTAL™.

Contrast agent solutions were prepared individually from stock solutions of cuttlefish ink for each imaging experiment. Stock cuttlefish ink solutions (50% w/v in 0.1 M PBS) were aliquoted and refrigerated. To prepare the contrast agent, an aliquot was diluted and mixed with corn starch to a final solution containing 5% ink w/v and 2% corn starch. It was briefly heated to boiling to achieve homogeneity. The spherical melanin nanoparticles within the contrast agent were previously characterized with transmission electron microscopy, which indicated a mean particle size of 125 nm from 500 nanoparticles; and dynamic light scattering, which showed a hydrodynamic radius of 266 nm with a polydispersity index of 0.116 (Lin et al. 2017).

### Volunteer

An experiment enrolled one 22-year old adult female with good oral hygiene. All work with human subjects was approved by the UCSD Institutional Review Board (170912) and conducted according to IRB guidelines. The participant gave written informed consent and teeth 7-10, 22-27 were imaged.

### Periodontal Probe Measurements

Pockets were measured with the Williams and Marquis probes (D. et al. 1997) by a licensed periodontist. The measurements were performed at the distobuccal, mesiobuccal, and buccal sites according to clinical convention (Savage et al. 2009). For distal and mesial measurements, the probe was inserted at a 10□ angle at the interproximal space between adjacent teeth. The buccal measurements were collected at the deepest point observed after walking the probe across the width of the pocket. Per standard clinical practice, measurements were rounded up to the nearest integer. For the Williams probe, all measurements <2 mm were recorded as 2 mm. For the Marquis probe, readings within the 3 mm intervals were estimated to be either in the lower 1.5 mm or upper 1.5 mm range and then rounded up to the nearest integer.

### Pocket Labeling

The subgingival pockets were labelled with ˜8 μL of contrast agent per tooth. A micropipette with a sterile 2-20 μL tip was placed in contact with the gingival sulcus and used to irrigate the region with contrast agent. Following imaging, the contrast agent was removed from the pockets by rinsing the mouth with water or gentle tooth brushing (<10 s).

### Imaging Procedure

Photoacoustic imaging was performed by pulsing light through two optical fiber bundles integrated with both sides of a rectangular, linear array transducer. The laser excitation (Q-switched Nd:YAG) used 5 ns pulses at 20 Hz (6 Hz frame rate). The ultrasound resolution was controlled by changing between three transducers: LZ-201 (center frequency=16 MHz), LZ-250 (center frequency=21 MHz), and LZ-550 (center frequency=40 MHz). Typical gains were 15 dB for photoacoustic signal and 10 dB for ultrasound. The human subject was seated in front of the imaging system and instructed to rest their chin on a flat surface in front of the transducer. Dental cheek retractors and a medical head immobilizer minimized vibrations that could perturb the imaging results. A layer of ultrasound gel was applied to the transducer, which was adjusted to 1 cm from the tooth. The 680 nm laser was initialized, and the stepper motor was scanned 17 mm (0.076 mm step size) to obtain a 3D PA-US image via maximum intensity projection (Fishman et al. 2006).

### Image Analysis and Statistics

Following raw data acquisition, sagittal cross-sections were examined in VEVO LAB™ software to determine the penetration of contrast agent and quantify pocket depths. From this view, the PA signal from the contrast agent could be traced from the gingival margin to its subgingival terminus. The pixel density of every image was standardized to the same physical dimensions, which allowed a line to be drawn parallel to the PA signal. This line represented the pocket depth and its magnitude was the pocket measurement.

To avoid bias during quantitative comparison to physical probing, sagittal planes were chosen the same way each time: for distobuccal and mesiobuccal sites, we chose the first 8 sagittal planes (0.6 mm-wide sections) with a measurable pocket depth on each lateral side of the tooth. These sections mimicked the diameters of the physical probes. For the buccal sites, 0.6 mm-wide sections were selected from the images at the deepest part of the pocket. This dimension mimicked the diameter of the physical probes and the typical procedure of recording the lowest number obtained by walking the probe across the pocket width. For replicate measurements, images were collected on nonconsecutive days across two weeks.

The buccal contours of the pocket geometry were mapped by averaging five separate imaging events. The width of the pocket consisted of dozens of sagittal planes. For each plane, two measurements were taken: the distance from the crown of the tooth to the gingival margin, and the distance from the gingival margin to the edge of photoacoustic signal (the pocket depth). Together, these measurements determined the top and bottom of the pocket for that plane. Once these measurements were taken for all planes, they were used to reconstruct a full map of the pocket to overlap on top of the ultrasound image.

### Results

### High-Resolution Ultrasound Imaging of Teeth and Soft Tissues

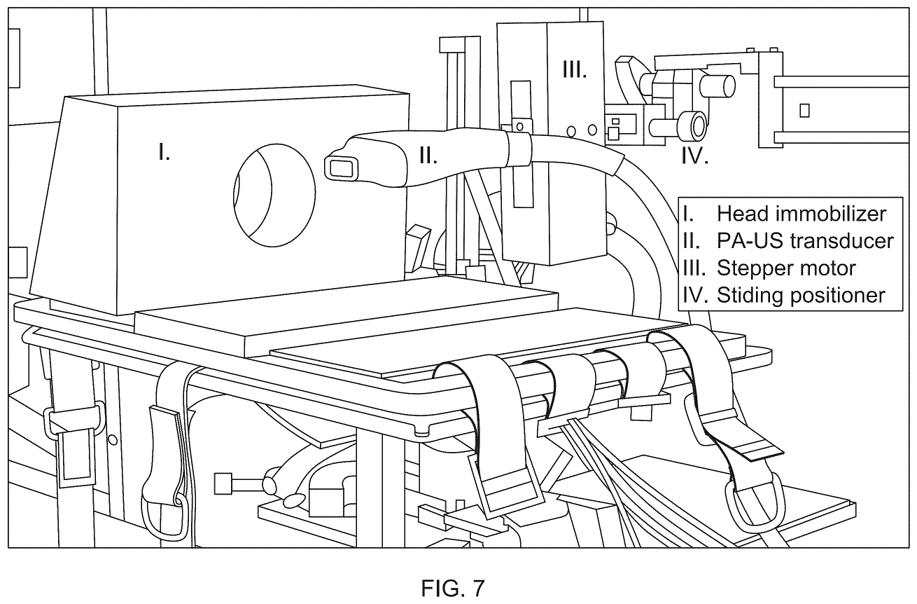

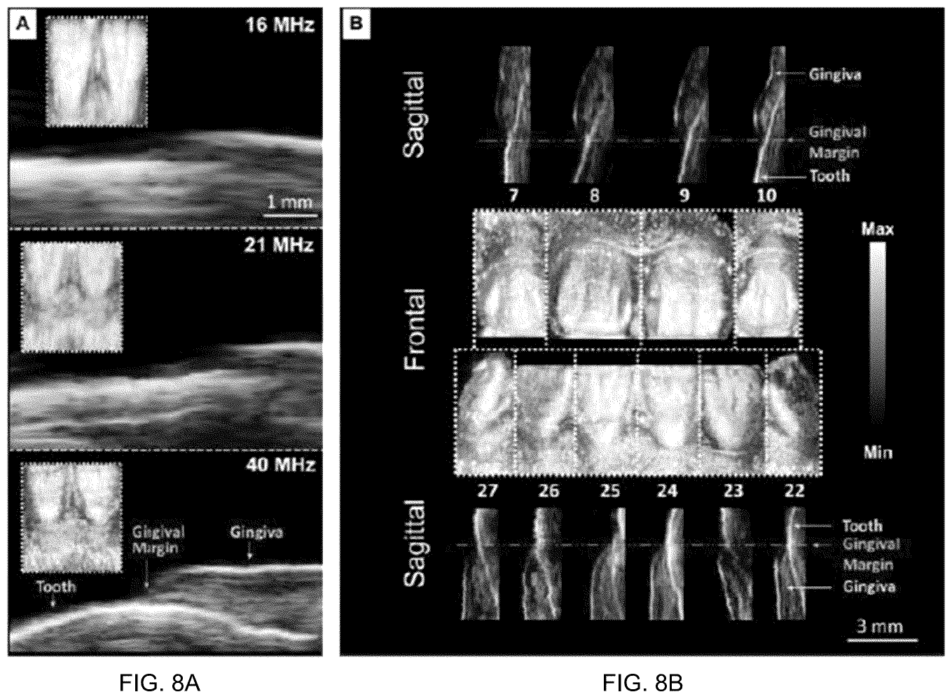

Photoacoustic and ultrasound images were collected by adapting a commercial PA-US system for human operation (FIG. 7). Ultrasound imaging was used to generate frontal and sagittal cross-sections of 10 teeth (tooth numbers 7-10, 22-27) and their associated soft tissues from a healthy volunteer (FIG. 8). They were labelled with the numbering system used by the American Dental Association (Harris 2005). These anterior teeth were imaged because the size of the transducer limited access to the posterior region of the oral cavity.

Increasing the ultrasound transducer frequency (FIG. 8A) significantly improved the ability to identify the gingival margin. The spatial resolution of the ultrasound mode was approximately 100 μm at 40 MHz and 300 μm for the photoacoustic mode (Lin et al. 2017; Moran et al. 2011). At 15 and 21 MHz, the gingival-tooth interface and subgingival tooth could not be identified. These features were clearly resolved at 40 MHz, and we used this frequency for all subsequent images. Frontal views captured surface features of the teeth and tissue, while sagittal views revealed the gingival margin, gingival thickness, and surface topology (FIG. 8B).

### Periodontal Labeling and Photoacoustic Imaging

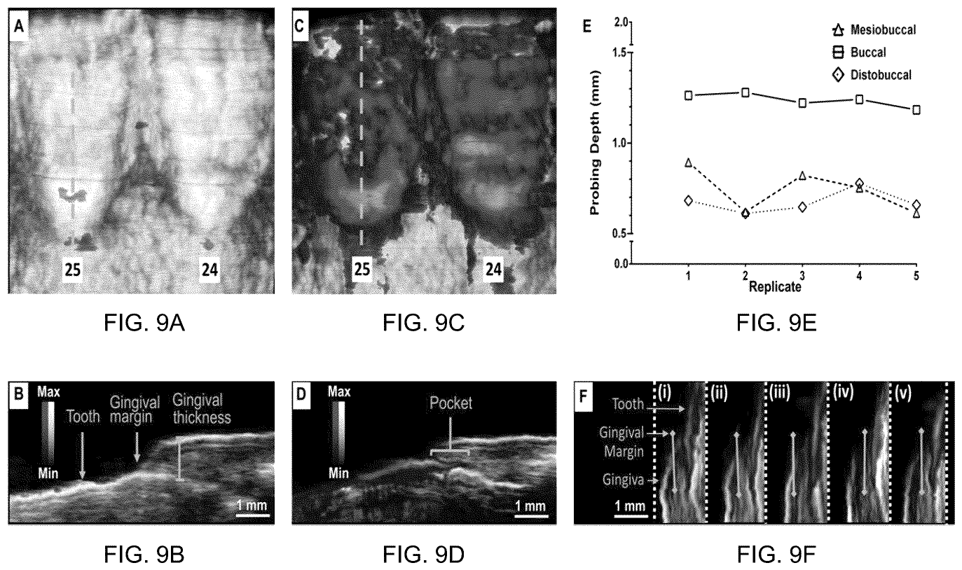

The contrast agent possessed broad photoacoustic absorption from 680 to 970 nm due to the presence of melanin nanoparticles (Fan et al. 2014; Lin et al. 2017). Following administration of contrast agent, PA-US images were collected, and the 680-nm signal was overlaid on ultrasound to measure the periodontal pocket depths. Frontal and sagittal views of the lower central incisors in FIG. 9A-D show the signal from the mid-buccal pocket after applying the contrast agent and imaging. The mid-buccal pocket depth of tooth number 25 was 1.31 mm (FIG. 9D); signal from the supragingival surface of the tooth did not contribute to the measurement. The gingival thickness could also be quantified. When measured at the mid-buccal plane, 2 mm below the gingival margin, the gingival thickness was 1.01±0.05 mm for tooth 25 and 0.97±0.04 mm for tooth 24 (n=5). These values matched the average thickness reported for a 16-24 year old age group (Vandana 2005).

To demonstrate reproducibility, five independent replicates were conducted by performing the entire labelling and imaging procedure from start to finish on different days. In FIG. 9E-F, the pocket depths are plotted across these replicates for buccal, mesiobuccal, and distobuccal planes. The means and standard deviations for these locations were 1.24±0.04 mm, 0.74±0.12 mm, and 0.68±0.06 mm respectively. These corresponded to relative standard deviations of 3%, 16%, and 8.8%.

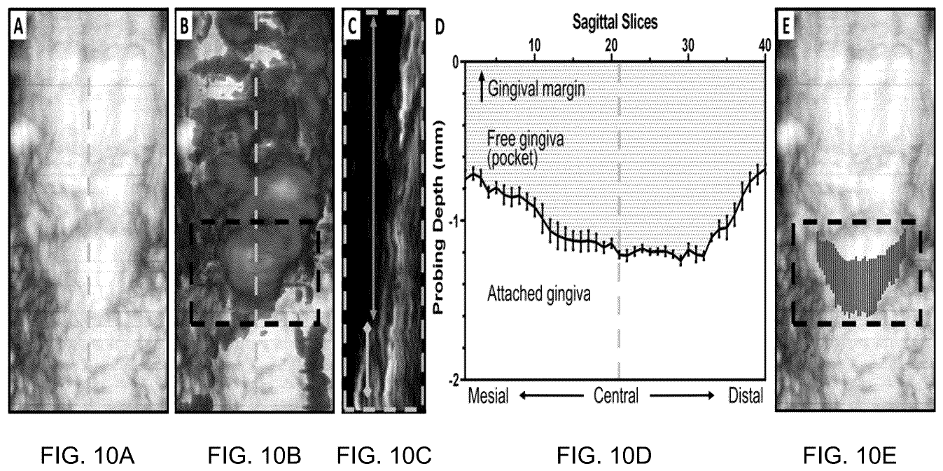

The pocket depth could be determined for a single plane, a region of planes, or the full buccal width of the pocket. The distance between each plane was 0.076 mm, corresponding to roughly 50-120 measurements per tooth. FIG. 4 shows the steps to construct a visualization of the full pocket overlaid on a frontal US image of the tooth. The measurements for these steps were performed five times (n=5) on a lower central incisor across its measurable width (FIG. 10A-B). The total mean for the full pocket width was 1.02±0.21 mm. The mean standard deviation of these measurements, i.e. the average of the standard deviations for every measurement plane across five replicates, was 0.104 mm, resulting in a relative standard deviation of 10.2% (FIG. 10D). Furthermore, the range of the measurements was 0.57 mm, reflecting the variation in pocket depth according to probing location. The distobuccal, buccal, and mesiobuccal regions of planes (0.6 mm wide each) had measurements of 0.84±0.17 mm, 1.15±0.05, and 0.79±0.05. FIG. 10C shows the two metrics required for each sagittal plane to generate the overlay in FIG. 10E: (1) the distance from crown to gingival margin and (2) the pocket depth. The final mapping of the pocket geometry is shown overlaid on the ultrasound image in FIG. 10E.

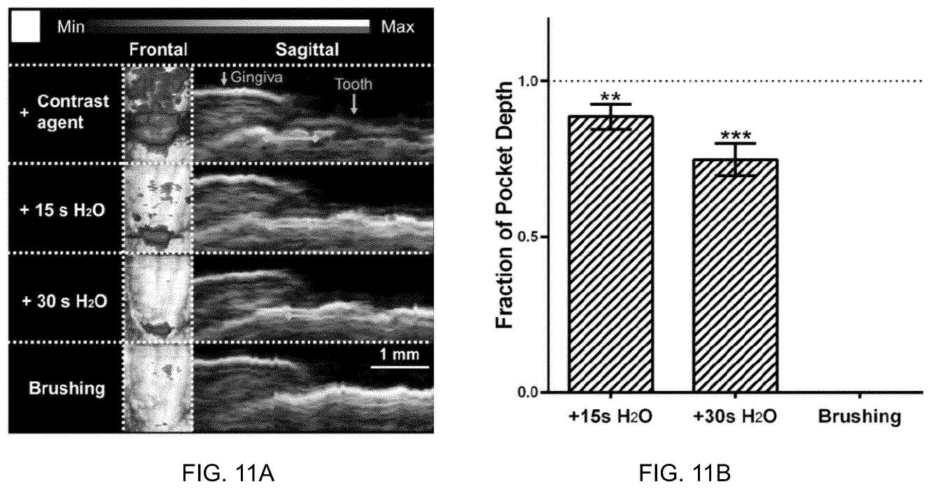

The persistence of contrast agent in the pockets was evaluated following oral rinses with water as well as brushing (FIG. 11). The reduction of pocket measurements was averaged from six representative planes across teeth 24 and 25 following 15 s rinsing, 30 s rinsing, and brushing; these steps resulted in 12.5%, 25.2%, and 100% reductions respectively when averaged across five replicates.

Finally, the pocket measurements were compared with the gold standard (Williams and Marquis) probes. The lowest possible measurement from the probes, as determined by a periodontist, was 2 mm (any lower values were rounded up); this value was recorded for the buccal pockets of teeth 24 and 25 with both probes. 1.34 mm and 1.15 mm (n=5) were measured for teeth numbers 24 and 25 with PA-US, respectively, which agreed with the gold standard measurements while providing more precision.

### Discussion

This experiment illustrates a new application of PA imaging for monitoring dental and periodontal health in humans by using a food-grade contrast agent. An example technique is ideal for imaging periodontal pockets, gingival thickness, and the interface between hard and soft tissues. It was determined that a 40 MHz ultrasound frequency provided sufficient resolution for discerning these features without sacrificing the requisite penetration depth. The US-only mode is particularly suited for applications that require knowledge of how a tooth is situated within the gingiva—one potential example would be diagnosis and monitoring of patients with delayed tooth eruption (Suri et al. 2004).

The flow of contrast agent into the gingival sulcus allowed the imaging and measurement of pocket depths. It was common for the agent to coat the majority of the tooth surface even when it was locally administered to the gingival margin (FIG. 9A); however, this did not adversely affect measurements. The beginning of the pocket could always be identified by overlaying the ultrasound image to reveal the gingival margin and gingival thickness (FIG. 9B). The imaging procedure took roughly 5 minutes per tooth but could be greatly improved with a mouthpiece transducer capable of scanning all teeth at once.

The PA imaging technique is highly reproducible (FIG. 9E, FIG. 9D) with relative standard deviations of 10%. This precision offers a major improvement over the Williams probe, which can only record integer values for pocket depths and has inter-operator error of up to 40% (Listgarden 1980). The periodontal pocket is a dynamic structure due to the constant battle between bacterial invasion and the immune response (Newman et al. 2011), and integers are a poor reflection of this process. In a typical oral exam, six measurements are taken per tooth (distolingual, lingual, mesiolingual, distobuccal, buccal, and mesiobuccal) to approximate the full profile of the pocket (Perry 2014). It was shown that photoacoustic imaging can be used to reveal the entirety of the buccal pocket with sub-millimeter (0.01 mm) precision (FIG. 10). The overlay in FIG. 10E was created manually but it represents a convenient visualization tool for clinicians; in the future, the conversion from raw data to final image with highlighted pocket would be performed by automated algorithms (Lehmann et al. 2002). Imaging the whole pocket over the patients' lifetimes has previously been impossible and could supersede periodontal charting—this practice could reveal the development of abnormalities that would otherwise be overlooked.

It was observed that contrast agent could be easily removed from the gingival sulcus after imaging (FIG. 11). Oral rinsing with water reduced the contrast agent both on the surface of the teeth and within the sulcus; a few seconds of normal brushing completely removed the agent. It was observed that the resulting PA-US measurements (#24=1.34 mm, #25=1.17 mm) were in good agreement with those acquired by the Williams and Marquis probes, which recorded 2 mm for both lower central incisors. The probes were not capable of distinguishing any values lower than 2 mm; in addition, they could only record integers and relied on subjective estimation. PA-US imaging removes ambiguity and improves the precision of measurements.

There were some limitations in this study. Due to the size of the PA-US transducer, only anterior teeth could be imaged. In principle, all 32 teeth and the periodontium can be imaged with this technique but the geometry of our PA-US transducer physically limited access. Fortunately, several groups have reported custom transducer geometries; a similar approach could overcome this limitation (Bell et al. 2014; Yang et al. 2015). The subject of the study had good oral hygiene and did not have deep (≥4 mm) pockets indicative of periodontal disease. However, Example 1, above, further demonstrates that deep pockets could be imaged in swine, a good model of the human gingiva (Wang et al. 2007). Finally, because the periodontal pocket is a dynamic structure, imaging and probing across different days may have contributed to small variations in measurements.

### Example 3: Photoacoustic-Ultrasound Imaging of Gingival Oxygen Gradients

This example shows an illustrative method of using photoacoustic imaging as a tool for measuring blood oxygen saturation in the gingiva. To collect the imaging data, a 40 MHz ultrasound transducer equipped with a near-infrared laser was placed over the lower central incisors of rabbits. A breathing mask was placed over the nose of the rabbits and the percent oxygen of the air was controlled.

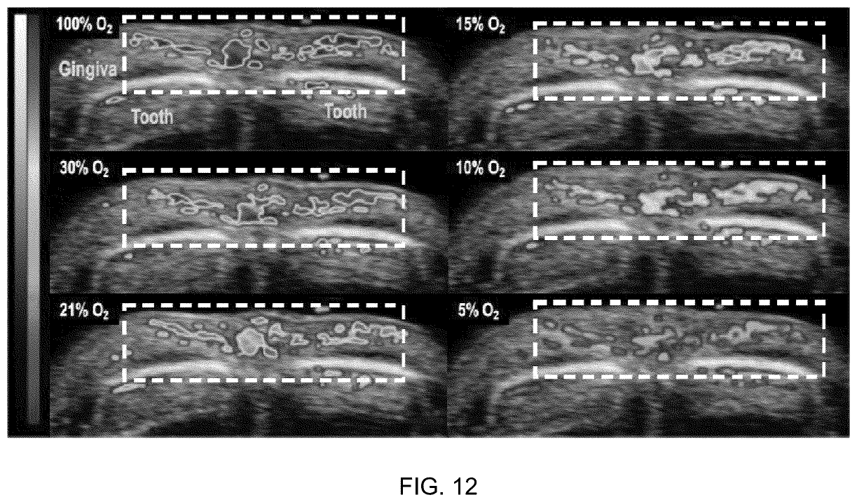

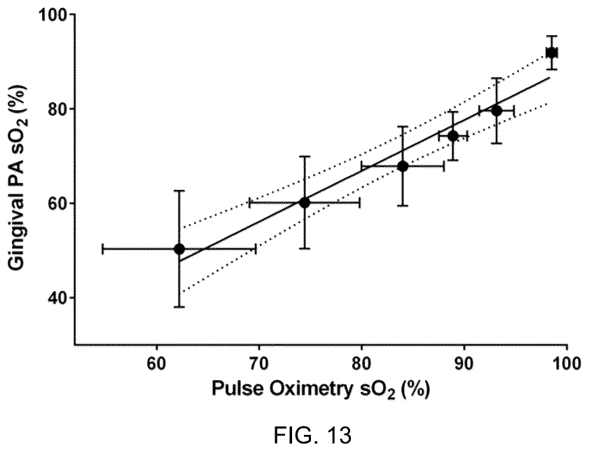

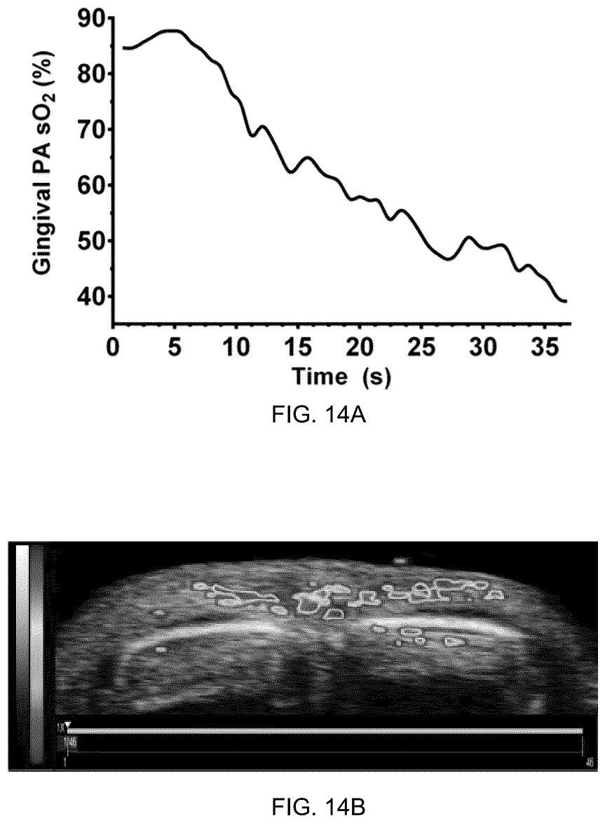

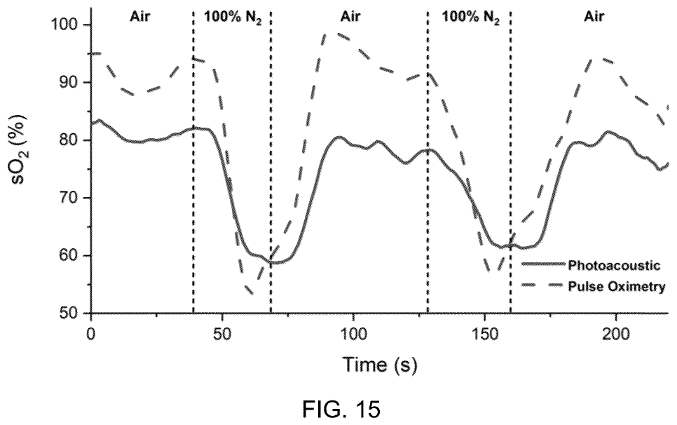

FIGS. 12 and 13 show the wide range of oxygen tensions detectable by local imaging and the correlation to pulse oximetry. FIGS. 14 and 15 highlight the abilities of photoacoustic imaging to track the gingival oxygen saturation in real-time, while correlating with pulse oximetry measurements. The use of photoacoustic imaging as provided herein also can map the degree of inflammation or infection that leads to dysregulated blood flow.

### References

Abramoff M D, Magelhaes P J, Ram S J. 2004. Image processing with ImageJ. Biphotonics Int. 11(7):36-42.
Arconada-Alvarez S J, Lemaster J E, Wang J, Jokerst J V. 2017. The development and characterization of a novel yet simple 3D printed tool to facilitate phantom imaging of photoacoustic contrast agents. Photoacoustics. 5:17-24.
Armitage et al. 1977. Microscopic evaluation of clinical measurements of connective tissue attachment levels. J Clin Periodontol. 4(3): 173-190.
Badersten A, Nilvéus R, Egelberg J. 1990. Scores of plaque, bleeding, suppuration and probing depth to predict probing attachment loss 5 years of observation following nonsurgical periodontal therapy. Journal of clinical periodontology. 17(2):102-107.
Balderas-López J A, et al. 1999. Thermal characterization of some dental resins using the photoacoustic phase lag discontinuities. Superficies y Vacio. 8:42-45.
Beard P. 2011. Biomedical photoacoustic imaging. Interface focus.rsfs20110028.
Bell M A L, Kuo N P, Song D Y, Kang J U, Boctor E M. 2014. In vivo visualization of prostate brachytherapy seeds with photoacoustic imaging. Journal of biomedical optics. 19(12):126011-126011.
Biddle A J, Palmer R M, Wilson R F, Watts T L. 2001. Comparison of the validity of periodontal probing measurements in smokers and non-smokers. J Clin Periodontol. 28(8): 806-812.
Bland J M, Altman D G. 1986. Statistical methods for assessing agreement between two methods of clinical measurement. Lancet. 1(8476):307-310.
Chaitanya L. 2014. Food coloring: the natural way. Res J Chem Sci. 4(2): 87-96.
Chan H-L, Sinjab K, Chung M-P, Chiang Y-C, Wang H-L, Giannobile W V, Kripfgans O D. 2017. Non-invasive evaluation of facial crestal bone with ultrasonography. PLoS One. 12(2):e0171237.
Chen et al. 2009. Adsorption of Pb(II) and Cd(II) by squid Ommastrephes bartrami melanin. Bioinorg Chem Appl. 7:901563.
Cheng et al. 2016. Noninvasive assessment of early dental lesion using a dual-contrast photoacoustic tomography. Sci Rep. 6:21798.
Cochran D L. 2008. Inflammation and bone loss in periodontal disease. Journal of periodontology. 79(8S):1569-1576.
D. SE, S. GG, A. P. 1997. In vitro accuracy and reproducibility of automated and conventional periodontal probes. Journal of Clinical Periodontology. 24(5):340-345.
D'aiuto F, Nibali L, Parkar M, Patel K, Suvan J, Donos N. 2010. Oxidative stress, systemic inflammation, and severe periodontitis. Journal of dental research. 89(11):1241-1246.
Darveau R P. 2010. Periodontitis: A polymicrobial disruption of host homeostasis. Nature Reviews Microbiology. 8(7):481.
Eke P I, Dye B, Wei L, Thornton-Evans G, Genco R. 2012. Prevalence of periodontitis in adults in the united states: 2009 and 2010. Journal of dental research. 91(10):914-920.
Emelianov S Y, Li P-C, O'Donnell M. 2009. Photoacoustics for molecular imaging and therapy. Physics today. 62(8):34.
Fan Q, Cheng K, Hu X, Ma X, Zhang R, Yang M, Lu X, Xing L, Huang W, Gambhir SS et al. 2014. Transferring biomarker into molecular probe: Melanin nanoparticle as a naturally active platform for multimodality imaging. Journal of the American Chemical Society. 136(43):15185-15194.
Fiorellini JP. 2016. A classification system for peri-implant diseases and conditions. Int J Periodontics Restorative Dent. 36(5):699-705.
Fishman E K, Ney D R, Heath D G, Corl F M, Horton K M, Johnson P T. 2006.
Volume rendering versus maximum intensity projection in ct angiography: What works best, when, and why. Radiographics. 26(3):905-922.
Fjærtoft M, Johannessen A C, Heyeraas K J. 1992. Micropuncture measurements of interstitial fluid pressure in normal and inflamed gingiva in rats. J Periodontal Res. 27(5):534-538.
Grossi S G, Dunford R G, Ho A, Koch G, Machtei E E, Genco R J. 1996. Sources of error for periodontal probing measurements. Journal of periodontal research. 31(5):330-336.
Hanli L, Boas D A, Yutao Z, Yodh A G, Chance B. 1995. Determination of optical properties and blood oxygenation in tissue using continuous NIR light. Phys Med Biol. 40(11):1983-1993.
Harris E F. 2005. Tooth-coding systems in the clinical dental setting. Den Anthr. 18(2):43-49.
Hefti A F. 1997. Periodontal probing. Crit Rev Oral Biol Med. 8(3):336-356.
Ho I T, Sessler J L, Gambhir S S, Jokerst J V. 2015. Parts per billion detection of uranium with a porphyrinoid-containing nanoparticle and in vivo photoacoustic imaging. Analyst. 140(11):3731-3737.
Holtfreter B, Albandar J M, Dietrich T, Dye B A, Eaton K A, Eke P I, Papapanou P N, Kocher T; Joint EU/USA Periodontal Epidemiology Working Group. 2015. Standards for reporting chronic periodontitis prevalence and severity in epidemiologic studies: proposed standards from the joint eu/USA periodontal epidemiology working group. J Clin Periodontol. 42(5):407-412.
Ju K-Y, et al. 2013. Bio-inspired, melanin-like nanoparticles as a highly efficient contrast agent for T1-weighted magnetic resonance imaging. Biomacromolecules. 14(10):3491-3497.
Karadottir H, Lenoir L, Barbierato B, Bogle M, Riggs M, Sigurdsson T, Crigger M, Egelberg J. 2002. Pain experienced by patients during periodontal maintenance treatment. Journal of periodontology. 73(5):536-542.
Khasnis S A, Kidiyoor K H, Patil A B, Kenganal S B. 2014. Vertical root fractures and their management. J Conserv Dent. 17(2):103-110.
Kornman K S. 2008. Mapping the pathogenesis of periodontitis: A new look. Journal of periodontology. 79(8S):1560-1568.
Lang N P, Adler R, Joss A, Nyman S. 1990. Absence of bleeding on probing: an indicator of periodontal stability. J Clin Periodontol. 17(10):714-721.
Lang et al. 1986. Bleeding on probing: a predictor for the progression of periodontal disease? J Clin Periodontol. 13(6):590-596.
Larsen et al. 2009. Probing pressure, a highly undervalued unit of measure in periodontal probing: a systematic review on its effect on probing pocket depth. J Clin Periodontol. 36(4):315-322.
Lee et al. 2017. Photoacoustic imaging of dental implants in a porcine jawbone ex vivo. Opt Lett. 42(9):1760-1763.
Lee Y T, Lee H C, Hu C J, Huang L K, Chao S P, Lin C P, Su E C Y, Lee Y C, Chen C C. 2017. Periodontitis as a modifiable risk factor for dementia: A nationwide population-based cohort study. Journal of the American Geriatrics Society. 65(2):301-305.
Lehmann T, Troeltsch E, Spitzer K. 2002. Image processing and enhancement provided by commercial dental software programs. Dentomaxillofacial Radiology. 31(4):264-272.
Lin C, Chen F, Hariri A, Chen C, Wilder-Smith P, Takesh T, Jokerst J. 2017. Photoacoustic imaging for noninvasive periodontal probing depth measurements. Journal of dental research.0022034517729820.
Listgarten M A. 1980. Periodontal probing: what does it mean? J Clin Periodontol. 7(3):165-176.
Mallidi S, Luke G P, Emelianov S. 2011. Photoacoustic imaging in cancer detection, diagnosis, and treatment guidance. Trends in biotechnology. 29(5):213-221.
Mariotti A, Hefti A. Defining periodontal health. BMC oral health; 2015: BioMed Central.
Mayfield L, Bratthall G, Attstrom R. 1996. Periodontal probe precision using 4 different periodontal probes. J Clin Periodontol. 23(2):76-82.
Michaud D, Kelsey K, Papathanasiou E, Genco C, Giovannucci E. 2016. Periodontal disease and risk of all cancers among male never smokers: An updated analysis of the health professionals follow-up study. Annals of Oncology. 27(5):941-947.
Moran C M, Pye S D, Ellis W, Janeczko A, Morris K D, McNeilly A S, Fraser H M. 2011. A comparison of the imaging performance of high resolution ultrasound scanners for preclinical imaging. Ultrasound in Medicine and Biology. 37(3):493-501.
Murphy et al. 2012. Corticotomy and stem cell therapy for orthodontists and periodontists: rationale, hypotheses, and protocol. In: Krishnan V, Davidovitch Z, editors. Integrated clinical orthodontics. Hoboken (NJ): John Wiley & Sons, Ltd. p. 392-421.
Nagarajan S, Zhang Y. 2011. Upconversion fluorescent nanoparticles as a potential tool for in-depth imaging. Nanotechnology. 22(39):395101.
Newman M G, Takei H, Klokkevold P R, Carranza F A. 2011. Carranza's clinical periodontology. Elsevier health sciences.
Nguyen K-C T, Le L H, Kaipatur N R, Major P W. 2016. Imaging the cemento-enamel junction using a 20-mhz ultrasonic transducer. Ultrasound Med Biol. 42(1):333-338.
Ntziachristos V, Razansky D. 2010. Molecular imaging by means of multispectral optoacoustic tomography (MSOT). Chem Rev. 110(5):2783-2794.
Perry D A, Beemsterboer P, Essex G. 2014. Periodontology for the dental hygienist. St. Louis (MO): Elsevier/Saunders.
Savage A, Eaton K A, Moles D R, Needleman I. 2009. A systematic review of definitions of periodontitis and methods that have been used to identify this disease. Journal of clinical periodontology. 36(6):458-467.
Schipper et al. 2007. Saliva as research material: biochemical, physicochemical and practical aspects. Arch Oral Biol. 52(12):1114-1135.
Slak et al. 2015. Assessment of gingival thickness using an ultrasonic dental system prototype: a comparison to traditional methods. Ann Anat. 199:98-103.
Stewart R, West M. 2016. Increasing evidence for an association between periodontitis and cardiovascular disease. Am Heart Assoc.
Suri L, Gagari E, Vastardis H. 2004. Delayed tooth eruption: Pathogenesis, diagnosis, and treatment. A literature review. American Journal of Orthodontics and Dentofacial Orthopedics. 126(4):432-445.
Vandana K L, Savitha B. 2005. Thickness of gingiva in association with age, gender and dental arch location. J Clin Periodontol. 32(7):828-830.
Vandenberghe B, Jacobs R, Bosmans H. 2010. Modern dental imaging: A review of the current technology and clinical applications in dental practice. European Radiology. 20(11):2637-2655.
Viator et al 2004. A comparative study of photoacoustic and reflectance methods for determination of epidermal melanin content. J Invest Dermatol. 122(6):1432-1439.
Walsh T F, Saxby M S. 1989. Inter- and intra-examiner variability using standard and constant force periodontal probes. J Clin Periodontol. 16(3):140-143.
Wang et al. 2016. A nanoscale tool for photo-acoustic-based measurements of clotting time and therapeutic drug monitoring of heparin. Nano Lett. 16(10):6265-6271.
Wang et al. 2007. The miniature pig: a useful large animal model for dental and orofacial research. Oral Dis. 13(6):530-537.
Wang L V, Hu S. 2012. Photoacoustic tomography: In vivo imaging from organelles to organs. science. 335(6075):1458-1462.
Wang S, Liu Y, Fang D, Shi S. 2007. The miniature pig: A useful large animal model for dental and orofacial research. Oral diseases. 13(6):530-537.
Weidmann et al. 2014. Depth determination of artificial periodontal pockets using cone-beam tomography and radio-opaque material: an in vitro feasibility study. Swiss Dent J. 124(4):406-415.
Yang J, Favazza C, Yao J. Three-dimensional photoacoustic and ultrasonic endoscopic imaging of two rabbit esophagi. SPIE BiOS; 2015: International Society for Optics and Photonics.
Yin et al. 2017. Degradable semiconducting oligomer amphiphile for ratiometric photoacoustic imaging of hypochlorite. ACS Nano. 11(4):4174-4182.
Zackrisson S, Van De Ven S, Gambhir S. 2014. Light in and sound out: Emerging translational strategies for photoacoustic imaging. Cancer research. 74(4):979-1004.

A number of embodiments of the invention have been described. Nevertheless, it can be understood that various modifications may be made without departing from the spirit and scope of the invention. Accordingly, other embodiments are within the scope of the following claims.

What is claimed is:

1. A product of manufacture or a dental device for imaging a periodontal tissue or a periodontium, wherein optionally the periodontal tissue or periodontium comprises an oral mucosa or gingiva, the method comprising:
a mouthpiece or mold capable of fitting over one or several teeth, or all teeth, in an arch (optionally a maxillary or mandibular arch, or both), and optionally also fitting over or covering (e.g., at least part of) the periodontal tissue or periodontium,
wherein optionally the mouthpiece or mold is manufactured to: image and collect signal(s) from a contrasting or imaging agent, and/or, to hold, carry and deliver to the contrasting agent or imaging agent to the periodontal tissue or periodontium, when the mouthpiece or mold is placed or fitted over the one or several or all teeth in the arch, optionally also covering periodontal tissue or periodontium or gingiva, and
one or more photoacoustic imaging sensors and corresponding transducers, wherein the one or more photoacoustic imaging sensors comprise ultrasound and/or optical imaging sensors capable of generating imaging data, and the ultrasound and optical imaging sensors are operatively linked to the corresponding ultrasound and optical transducers, and the ultrasound and/or optical transducer is capable of transmitting the imaging data to a remote receiver,
wherein the one or more photoacoustic imaging sensors can image the periodontium or periodontal tissue, or a periodontal pocket, and generate imaging data,
and optionally the imaging data from the one or more photoacoustic imaging sensors is capable of being converted to: an image of the periodontium or periodontal tissue, or an image of the periodontal pocket, or a periodontal probing depth image or measurement.

2. The product of manufacture or dental device of claim 1, wherein the mouthpiece or mold comprises an outer body shaped to fit over (or cover) the one or several teeth, or all teeth, optionally also including fitting over or covering periodontium or gingiva, in an arch; and/or
wherein the one or more photoacoustic imaging sensors and corresponding transducers comprises: one or more ultrasonic transducers disposed within or on the outer body.

3. The product of manufacture or dental device of claim 2, wherein the one or more ultrasonic transducers are disposed within or on the outer body to substantially align with the one or several teeth, or all teeth, in an arch when the outer body is inserted into a patient's mouth.

4. The product of manufacture or dental device of claim 2, wherein the one or more photoacoustic imaging sensors and corresponding transducers further comprises one or more light generators, optionally light-emitting diodes (LEDs), disposed within or on the outer body.

5. The product of manufacture or dental device of claim 4, wherein the one or more light generators are received within openings in a gum-facing surface of the outer body.

6. The product of manufacture or dental device of claim 1, further comprising a wired or wireless transmittal device to transmit the imaging data to a computer and/or a database, or to an electronic medical record or to a computer hard disk or computer memory, or to a cloud-based storage source,
and optionally the ultrasound and/or optical transducers operatively linked to their corresponding ultrasound and optical imaging sensors are operatively linked to the wired or wireless transmittal device.

7. The produce of manufacture of dental device of claim 1, wherein the mouthpiece or mold comprises an outer body shaped to fit over (or cover) the one or several teeth, or all teeth, optionally also including fitting over or covering periodontium or gingiva, in an arch; and further comprising a non-transitory memory stored within an outer body of the mouth and configured for storing the imaging data.

8. The product of manufacture or dental device of claim 1, further comprising an acoustic coupling medium or a gel, or equivalent, to transmit acoustic pressure waves from an oral mucosa, a gum, a tooth or a periodontal pocket, to the acoustic transducers in the product of manufacture or dental device.

9. The product of manufacture or dental device of claim 1, further comprising a photoacoustic imaging agent,
wherein optionally photoacoustic imaging agent comprises a food based imaging agent, or optionally comprises a food-grade squid ink or an equivalent optionally also comprising a cornstarch or an equivalent,
wherein optionally photoacoustic imaging agent comprises an optical absorber, optionally a strong optical absorber, or optionally an optical absorber comprising a melanin.

10. The product of manufacture or dental device of claim 9, wherein the photoacoustic imaging agent comprises melanin nanoparticles.

11. A method for:
measuring or detecting an inflammation or a bacterial infection, or a hypoxia or blood flow (e.g., the amount of blood flow), in an oral mucosa, a periodontal pocket or in a periodontium (e.g., gingiva),
image a periodontium or periodontal tissue, or a periodontal pocket,
measuring the depth of a periodontal pocket,
measuring or detecting calcification, calculus (or tartar, or hardened dental plaque) or dental stains,
measuring or detecting gingival thickness, and/or
measuring or detecting peri-implantitis, comprising:
(a) providing a product of manufacture or a dental device of any of the preceding claims;
(b) providing a photoacoustic imaging agent, and/or an acoustic coupling medium or a gel, or equivalent,
wherein optionally photoacoustic imaging agent comprises a food based imaging agent, or optionally comprises a food-grade squid ink or an equivalent optionally also comprising a cornstarch or an equivalent,
wherein optionally photoacoustic imaging agent comprises an optical absorber, optionally a strong optical absorber, or optionally an optical absorber comprising a melanin,
(c) placing the photoacoustic imaging agent and/or acoustic coupling medium or a gel, or equivalent on the periodontium, gingiva, or the oral mucosa of an individual, optionally a dental patient, or placing the photoacoustic imaging agent and/or acoustic coupling medium or a gel, or equivalent in the product of manufacture or the dental device such that the photoacoustic imaging agent comes into contact with the periodontium or oral mucosa,
(d) taking a photoacoustic and/or acoustic coupling medium or a gel, or equivalent imaging of the periodontium or oral mucosa with the one or more photoacoustic imaging sensors to generate ultrasound and/or optical imaging data, and
optionally, (e) saving the ultrasound and/or optical imaging data in the product of manufacture or the dental device, wherein the product of manufacture or a dental device comprises a data memory storage, or transmitting the ultrasound and optical imaging data to a computer, a memory and/or a database.

12. The method of claim 1, further comprising a wired or wireless transmittal device to transmit the imaging data to a computer and/or a database, or to an electronic medical record or to a computer hard disk or computer memory, or to a cloud-based storage source.

13. The method of claim 1, wherein the ultrasound and/or optical imaging data from the one or more photoacoustic imaging sensors is capable of being converted to: an image of the periodontium or periodontal tissue, or an image of a periodontal pocket, or a periodontal probing depth measurement, or the ultrasound and optical imaging data from the one or more photoacoustic imaging sensors is converted to: an image of the periodontium or periodontal tissue, or an image of a periodontal pocket, or a periodontal probing depth measurement.

14. Use of a product of manufacture or dental device of any of the preceding claims for
measuring or detecting an inflammation or a bacterial infection, or a hypoxia or blood flow (e.g., the amount of blood flow), in an oral mucosa, a periodontal pocket or in a periodontium (e.g., gingiva),
image a periodontium or periodontal tissue, or a periodontal pocket,
measuring the depth of a periodontal pocket,
measuring or detecting calcification, calculus (or tartar, or hardened dental plaque) or dental stains,
measuring or detecting gingival thickness, and/or
measuring or detecting peri-implantitis.

15. A kit comprising a product of manufacture or dental device of any of the preceding claims, and optionally further comprising a photoacoustic imaging agent and/or acoustic coupling medium or a gel, or equivalent,
wherein optionally photoacoustic imaging agent comprises a food based imaging agent, or optionally comprises a food-grade squid ink or an equivalent optionally also comprising a cornstarch or an equivalent,
wherein optionally photoacoustic imaging agent comprises an optical absorber, optionally a strong optical absorber, or optionally an optical absorber comprising a melanin,
and optionally further comprising instructions for practicing a method of any of the preceding claims.

16. The kit of claim 15, further comprising:
an applicator for applying the photoacoustic imaging agent to a patient's mouth, wherein optionally the applicator is a syringe, and optionally the syringe is preloaded with the photoacoustic imaging agent.

17. The kit of claim 15, further comprising:
a processing device for receiving the imaging data and generating at least one image of the periodontal tissue or periodontium, and optionally further comprising an imaging device for displaying the image of the periodontal tissue or periodontium.

18. The kit of claim 17, wherein said processing device comprises:
a processor;
a wired or wireless interface for operably communicating, directly or indirectly, with the photoacoustic imaging sensors;
computer-executable instructions stored on a non-transitory memory for causing the processor to receive the image data and generate the at least one image of the periodontal tissue or periodontum.

19. The kit of claim 17, wherein said processing device is further configured to:
generate an additional ultrasound image of the periodontal tissue or periodontum, or portion thereof; and
overlay one of the generate the at least one image of the periodontal tissue or periodontum or the generated additional ultrasound image, or vice versa, to produce a composite image.

20. The kit of claim 17, further comprising:
an additional processing device for receiving the imaging data directly from the photoacoustic imaging sensors, storing at least a portion of the receiving imaging data, and transmitting the stored imaging data, wired and/or wirelessly, to the processing device.

21. A product of manufacture or a dental device for use in:
measuring or detecting an inflammation or a bacterial infection, or a hypoxia or blood flow (e.g., the amount of blood flow), in an oral mucosa, a periodontal pocket or in a periodontium (e.g., gingiva),
image a periodontium or periodontal tissue, or a periodontal pocket,
measuring the depth of a periodontal pocket,
measuring or detecting calcification, calculus (or tartar, or hardened dental plaque) or dental stains,
measuring or detecting gingival thickness, and/or
measuring or detecting peri-implantitis,
wherein the product of manufacture or the dental device comprises a product of manufacture or a dental device as set forth in any of the preceding claims.
